(19) United States
(12) Patent Application Publication
(10) Pub. No.: US 2025/0170065 A1
(43) Pub. Date: May 29, 2025

(54) THERAPEUTIC COMPOUNDS, FORMULATIONS, AND USE THEREOF

(71) Applicant: Tvardi Therapeutics, Inc., Sugar Land, TX (US)

(72) Inventors: Imran Alibhai, Sugar Land, TX (US); Sofia de Achaval, Missouri City, TX (US); Ralph Caricofe, Beaufort, NC (US); L. Graham Whitesell, Jr., Durham, NC (US); Ian Smart, Ellicott City, MD (US)

(21) Appl. No.: 18/842,626

(22) PCT Filed: Mar. 1, 2023
(86) PCT No.: PCT/US2023/063520

§ 371 Date: Aug. 29, 2024

Related U.S. Application Data
(60) Provisional application No. 63/268,726, filed on Mar. 1, 2022.

(51) Int. Cl. A61K 9/14; A61K 9/20; A61K 31/18

(52) U.S. Cl. CPC A61K 9/146; A61K 9/2013; A61K 9/2027; A61K 9/2054; A61K 31/18

(57) ABSTRACT

Provided herein are solid amorphous dispersions, compositions, and solid oral dosage forms comprising a compound of Formula (I). In specific instances, such compositions comprise a polymer excipient, and optionally a crystallization inhibiting polymer. In some instances, such compositions are useful for the treatment of fibrosis, cancer, and/or inflammatory disease/disorder.

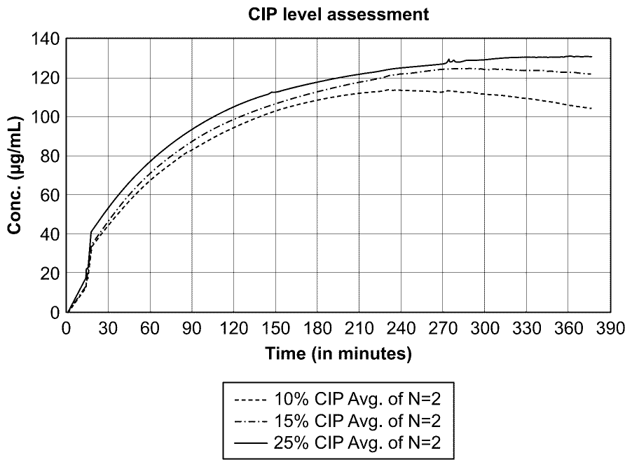

### CROSS-REFERENCE

This application is the U.S. National Stage entry of International Application No. PCT/US2023/063520, filed Mar. 1, 2023, which claims the benefit of and priority to U.S. Provisional Patent Application No. 63/268,723, filed Mar. 1, 2022, each of which is incorporated by reference in its entirety herein.

### SUMMARY

Disclosed herein, in certain embodiments, are solid amorphous dispersions comprising a compound of Formula (I) and a polymer excipient, methods of making said dispersions, compositions comprising said dispersions, oral dosage forms comprising said compositions, as well as methods of treating diseases or disorders (e.g., cancer, fibrosis, inflammatory diseases or disorders) comprising administering oral dosage forms comprising said compositions.

Disclosed herein, in certain embodiments, are solid amorphous dispersions comprising:
a) a compound of formula (I):

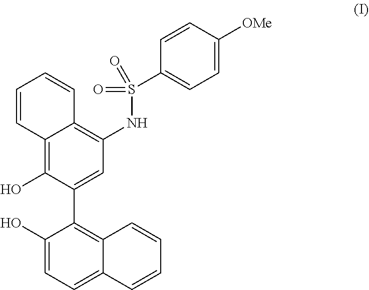 [SMILES: COc1ccc(S(=O)(=O)Nc2cc(-c3c(O)ccc4ccccc34)c(O)c3ccccc23)cc1]

or a pharmaceutically acceptable salt thereof; and
b) a polymer excipient.

Disclosed herein, in certain embodiments, are solid amorphous dispersions comprising:
a) a compound of formula (I):

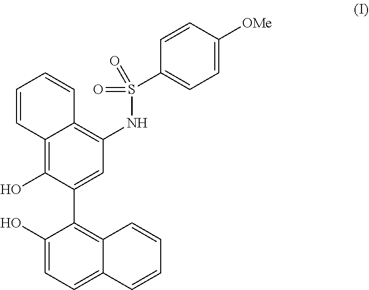 [SMILES: COc1ccc(S(=O)(=O)Nc2cc(-c3c(O)ccc4ccccc34)c(O)c3ccccc23)cc1]

or a pharmaceutically acceptable salt thereof, in an amount of about 40% w/w to about 60% w/w of the dispersion; and
b) a polymer excipient in an amount of about 40% to about 60% w/w of the dispersion.

Disclosed herein, in certain embodiments, are solid amorphous dispersions comprising:
a) at least 150 mg of a compound of formula (I):

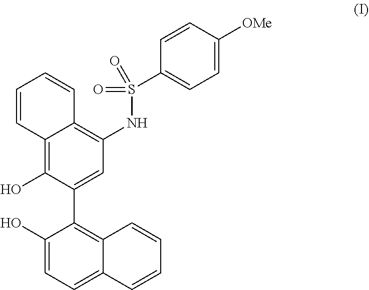 [SMILES: COc1ccc(S(=O)(=O)Nc2cc(-c3c(O)ccc4ccccc34)c(O)c3ccccc23)cc1]

or a pharmaceutically acceptable salt thereof; and
b) a polymer excipient.

Disclosed herein, in certain embodiments, are compositions comprising:
a) a solid amorphous dispersion comprising:
   i) at least 150 mg of a compound of formula (I):

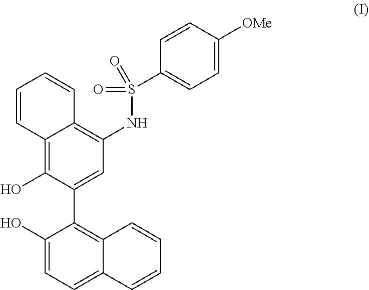 [SMILES: COc1ccc(S(=O)(=O)Nc2cc(-c3c(O)ccc4ccccc34)c(O)c3ccccc23)cc1]

or a pharmaceutically acceptable salt thereof; and
ii) a polymer excipient in an amount of about 20-35% w/w of the composition;
b) a crystallization inhibiting polymer in an amount of about 15-35% w/w of the composition;
c) a diluent or filler in an amount of about 5-25% of the composition;
d) a disintegrant in an amount of about 1-8% w/w of the composition; and
e) a lubricant in an amount of about 0.1-3% w/w of the composition.

Disclosed herein, in certain embodiments, are compositions comprising:
a) a solid amorphous dispersion comprising:
   i) at least 150 mg of a compound of formula (I):

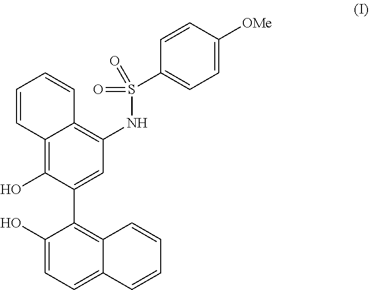 [SMILES: COc1ccc(S(=O)(=O)Nc2cc(-c3c(O)ccc4ccccc34)c(O)c3ccccc23)cc1]

or a pharmaceutically acceptable salt thereof; and
ii) a polymer excipient in an amount of about 15-25% w/w of the composition;
b) a crystallization inhibiting polymer in an amount of about 15-25% w/w of the composition;
c) a diluent or filler in an amount of about 20-35% of the composition;
d) a disintegrant in an amount of about 3-8% w/w of the composition; and
e) a lubricant in an amount of about 1-3% w/w of the composition.

Disclosed herein, in certain embodiments, are solid oral dosage forms comprising a compound of formula (I).

Disclosed herein, in certain embodiments, are solid oral dosage forms comprising a composition disclosed herein.

Disclosed herein, in certain embodiments, are methods of treating cancer in an individual in need thereof, the method comprising administering to the individual the oral dosage form disclosed herein.

Disclosed herein, in certain embodiments, are methods of treating fibrosis in an individual in need thereof, the method comprising administering to the individual the oral dosage form disclosed herein.

Disclosed herein, in certain embodiments, are methods of treating an inflammatory disease or disorder in an individual in need thereof, the method comprising administering to the individual the oral dosage form disclosed herein.

Disclosed herein, in certain embodiments, are methods of treating chemotherapy-induced peripheral neuropathy, diabetic neuropathy, or familial amyloid polyneuropathy in an individual in need thereof, the method comprising administering to the individual the oral dosage form disclosed herein.

Disclosed herein, in certain embodiments, are methods of treating cachexia in an individual in need thereof, the method comprising administering to the individual the oral dosage form disclosed herein.

Disclosed herein, in certain embodiments, are methods of treating anaphylaxis in an individual in need thereof, the method comprising administering to the individual the oral dosage form disclosed herein.

Other objects and advantages will become apparent to those skilled in the art from a consideration of the ensuing Detailed Description, Examples, and Claims.

### BRIEF DESCRIPTION OF THE DRAWINGS

The FIGURE shows the kinetic solubility of TTI-101 compositions with 10%, 15%, or 25% CIP in FaSSIF (pH 6.5) monitored for 6 hours.

### DETAILED DESCRIPTION

As generally described herein, the present disclosure, in part, provides solid amorphous dispersions comprising a compound of Formula (I) and a polymer excipient, methods of making said dispersions, compositions comprising said dispersions, oral dosage forms comprising said compositions, as well as methods of treating diseases or disorders (e.g., cancer, fibrosis, inflammatory diseases or disorders) comprising administering oral dosage forms comprising said compositions.

### Definitions

As used herein the specification, “a” or “an” may mean one or more. As used herein, when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. Some embodiments of the invention may consist of or consist essentially of one or more elements, method steps, and/or methods of the invention. It is contemplated that any method, compound, or composition described herein can be implemented with respect to any other method, compound, or composition described herein.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺ (C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “oral dosage form” as used herein and unless otherwise indicated, refers to a pharmaceutical composition that has been formulated or otherwise prepared for oral administration, such as in a discrete form.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In some embodiments, the subject is a human. In some embodiments, the subject is a non-human animal. The terms “human,” “patient,” “subject,” and “individual” are used interchangeably herein. None of these terms require the active supervision of medical personnel.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or reverses or slows the progression of the disease, disorder or condition (also “therapeutic treatment”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. A “therapeutically effective amount” of a compound is an amount sufficient to provide a therapeutic benefit (e.g., treating, preventing, and/or ameliorating cancer in a subject, or inhibiting protein-protein interactions mediated by an SH2 domain in a subject, at a reasonable benefit/risk ratio applicable to any medical treatment) in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent. A “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent. A “prophylactic treatment” contemplates an action that occurs before a subject begins to suffer from the specified disease, disorder or condition.

### Compositions, Formulations, Oral Dosage Forms

The compound of Formula (I) (also referred to as TTI-101) is highly insoluble (“brick dust”). It is insoluble in aqueous solutions and has relatively low solubility in most solvents. Furthermore, the compound of Formula (I) has a crystalline structure with high crystal-lattice energy, contributing to rapid precipitation or crystallization of the compound from solution. Its low solubility and high crystallinity have contributed to various difficulties in preparing formulations that are suitable for administration in human subjects. Current formulations are liquid formulations delivered via capsules. For example, a formulation of the compound of formula (I) dissolved in 60:40 Labrasol®/PEG400 and encapsulated into a hard gelatin capsule (unit dose: 36 mg), while showing encouraging results in 3 dose cohorts, was prevented from advancing to a fourth patient cohort (25.6 mg/kg/day as 12.8 mg/kg twice daily (BID) doses) because of an unacceptably high pill burden. For example, a 70 kg subject in Cohort 4 would require 60 capsules per day split into BID doses. Other oral dosage forms comprising the compound of formula (I) (e.g., as a self-emulsifying drug dispersion, e.g., comprising Kolliphor® RH 40 (PEG-40 hydrogenated castor oil), PEG600, Polysorbate 20, Labrasol®, and citric acid) are able to reduce pill burden compared to the two-component system (e.g., 60 capsules per day of the two-component system to 22 capsules per day in the same Cohort 4 with the self-emulsifying drug dispersion); however, such formulations still have limited drug loading per single oral dosage form and may require, for example, refrigerated (e.g., 2-8° C.) storage conditions.

Disclosed herein, in certain embodiments, are solid oral dosage forms comprising a solid amorphous dispersion comprising a compound of Formula (I):

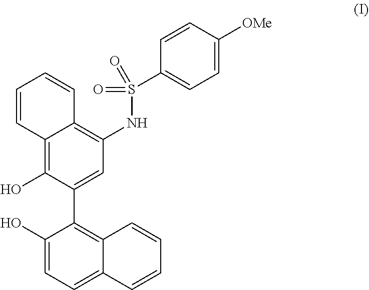 [SMILES: COc1ccc(S(=O)(=O)Nc2cc(-c3c(O)ccc4ccccc34)c(O)c3ccccc23)cc1]

or a pharmaceutically acceptable salt thereof, and a pharmaceutically acceptable excipient that provide high loading of a compound of Formula (I), good ability to provide for high bioavailability of a compound of Formula (I), good stability (e.g., chemical and/or physical stability), and/or reduce pill burden for individuals receiving therapies involving the administration of a compound of Formula (I).

The excipient or combination of excipients disclosed herein provide(s) improved solubility of a compound of Formula (I), improved physical stability (e.g., low crystallinity, good solubility and/or dispersion of a compound of Formula (I)), improved chemical stability of a compound of Formula (I), improved (e.g., oral) bioavailability of a compound of Formula (I), no leakage of any liquid (e.g., as compared to banded capsules), longer shelf life, favorable storage conditions (e.g., improved storage conditions of the compositions, formulations, and oral dosage forms at ambient conditions, e.g., requiring less protection from oxygenation and/or humidity), and/or desirable or therapeutic effect, with a manageable (e.g., fewer than 10 pills per day (e.g., 8 pills or fewer per day), or other amount described herein) and/or reduced pill burden (e.g., relative to two component excipient systems described herein and self-emulsifying drug dispersion systems described herein). In some embodiments, the excipient or combination of excipients is/are suitable for maintaining or stabilizing the compound of Formula (I) in an amorphous state (e.g., as a stabilized amorphous dispersion). In some embodiments, the compositions disclosed herein reduce or prevent the crystallization of the compound of Formula (I) when the compound is released in the gastrointestinal (GI) tract.

In some embodiments, the dispersions, compositions, and oral dosage forms described herein comprise a compound of Formula (I) as a pharmaceutically acceptable salt, a hydrate, or a solvate.

In some embodiments, a composition disclosed herein is set forth as in Table 1 (e.g., wherein the total wt. % in the composition does not exceed 100%).

TABLE 1
Exemplary Solid Amorphous Dispersion Compositions

| | Formulation | Formulation | Formulation | Formulation | Formulation |
|---|---|---|---|---|---|
| Component | 1 (% w/w) | 2 (% w/w) | 3 (% w/w) | 4 (% w/w) | 5 (% w/w) |
| Polymer | 10-40 | 20-30 | 30-70 | 40-60 | 50 |
| excipient | | | | | |
| Crystallization | 10-40 | 20-30 | — | — | — |
| inhibiting | | | | | |
| polymer | | | | | |
| API (TTI-101) | 30-70 | 40-60 | 30-70 | 40-60 | 50 |

The solid amorphous dispersions described herein are prepared using spray-dried dispersion (SDD) technique. SDD produces a single-phase, amorphous molecular dispersion (a solid solution) of the API in a polymer matrix. In general, SDDs are obtained by dissolving an API (e.g., TTI-101) and a polymer excipient(s) in a solvent(s) and then spray drying the solution. Key to the process is identifying a polymer excipient(s) and process conditions that result in the solvent quickly evaporating from the droplets of the spray solution of TTI-101 and polymer excipient(s), allowing insufficient time for phase separation of the TTI-101 or polymer excipient(s) within the solution or crystallization of the TTI-101 in the solution.

In some embodiments, the solid amorphous dispersions prepared using spray-dried dispersion (SDD) techniques described herein provide for reduced pill burden. In some embodiments, formulations described herein prepared using the SDD technique results in reduced pill burden compared to a formulation prepared using a self-emulsifying drug dispersion (SEDD) technique. For example, three times as many pills prepared using the SEDD technique (e.g., Formulation 2) would be needed as compared to a formulation prepared using the SDD technique (e.g., Formulation 3). In some embodiments, a number of pills needed of Formulation 3 is 15 pills or fewer. In some embodiments, a number of pills needed of Formulation 3 is 10 pills or fewer. In some embodiments, a number of pills needed of Formulation 3 is 8 pills or fewer. In some embodiments, a number of pills prepared by the methods described herein is 6 pills or fewer. In some embodiments, a number of pills needed of Formulation 3 is 4 pills or fewer. In some embodiments, a number of pills needed of Formulation 3 is 3 pills or fewer. In some embodiments, a number of pills needed of Formulation 3 is 2 pills or fewer.

In some embodiments, formulations described herein prepared using the SDD technique (e.g., Formulation 3) lowers the pill burden required to achieve a therapeutic dose of TTI-101 as compared to a formulation prepared using the SEDD technique (e.g., Formulation 2) or Formulation 1. In some embodiments, 1.5× more TTI-101 is delivered from Formulation 3 as compared to Formulation 1 or Formulation 2. In some embodiments, 2.0× more TTI-101 is delivered from Formulation 3 as compared to Formulation 1 or Formulation 2. In some embodiments, 2.5× more TTI-101 is delivered from Formulation 3 as compared to Formulation 1 or Formulation 2. In some embodiments, 3.0× more TTI-101 is delivered from Formulation 3 as compared to Formulation 1 or Formulation 2. In some embodiments, 4× more TTI-101 is delivered from Formulation 3 as compared to Formulation 1 or Formulation 2. In some embodiments, 5× more TTI-101 is delivered from Formulation 3 as compared to Formulation 1 or Formulation 2. For example, Formulation 3 can deliver 200 mg of TTI-101 per unit versus 80 mg of TTI-101 per unit when Formulation 2 is used and 30 mg of TTI-101 per unit when Formulation 1 is used.

In some embodiments, the solvent employed in the SDD technique to dissolve the TTI-101 is selected from: dichloromethane, methanol, ethanol, acetone, tetrahydrofuran, ethyl acetate, water, or a mixture thereof. In some embodiments, the solvent employed in the SDD technique for preparing a solid amorphous dispersion comprising TTI-101 and a polymer excipient is a mixture of acetone:water.

In some embodiments, the polymer excipient is one or more celluloses or derivatives thereof (e.g., microcrystalline cellulose, carboxymethylcellulose, hydroxypropyl methylcellulose (HPMC) or hypromellose, hydroxypropyl methylcellulose acetate succinate (HPMCAS) or hypromellose acetate succinate (e.g., HPMCAS-LG, HPMCAS-MG, HPMCAS-HG), HPMC phthalate (HPMCP) (e.g., HPMCP-HP55, HPMCP-HP55S), Methocel™ (e.g., Methocel™ E3 (e.g., Methocel™ E3LV))), hydroxypropyl cellulose, hydroxyethylcellulose, ethylcellulose, and cellulose acetate phthalate), polyacrylates (e.g., polymethacrylates (e.g., copolymers comprising methacrylic acid and methyl methacrylate, copolymers comprising methacrylic acid and ethylacrylate, copolymer comprising N,N-dimethylaminoethyl methacrylate, methylmethacrylate, and butylmethacrylate (e.g., Eudragit®, (e.g., Eduragit® EPO, Eudragit® L30 D-55, Eudragit® L100, Eudragit® L100-55)))), polyvinyl pyrrolidones (PVP), polyvinyl pyrrolidone vinyl acetates (PVPVA), other copolymers (e.g., copolymers comprising polyethylene glycol, polyvinylcaprolactam, and polyvinylacetate (e.g., polyvinyl acetate phthalate (PVAP); polyvinyl caprolactam-polyvinyl acetate-polyethylene glycol graft copolymer)) (e.g., Soluplus®), or copolymers comprising vinyl acetate and N-vinyl-2-pyrrolidone (e.g., Plasdone™ (e.g., Plasdone™ S630 (e.g., Plasdone™ S630 Ultra)), or mixtures thereof.

In some embodiments, the polymer excipient is selected from: hydroxypropyl methylcellulose (HPMC), a copolymer comprising methacrylic acid, a copolymer comprising vinyl acetate, and N-vinyl-2-pyrrolidone, and combinations thereof.

In some embodiments, the polymer excipient is hydroxypropyl methylcellulose acetate succinate (HPMC AS). In some embodiments, the HPMC AS is HPMC AS L grade (HPMC AS-L).

In some embodiments, the polymer excipient is a copolymer comprising methacrylic acid and ethylacrylate, a copolymer comprising methacrylic acid and methyl methacrylate, or a copolymer comprising N,N-dimethylaminoethyl methacrylate, methylmethacrylate, and butylmethacrylate. In some embodiments, the polymer excipient is a copolymer comprising methacrylic acid and ethylacrylate. In some embodiments, the polymer excipient is a copolymer of about 1.4:1 to about 1:1.4 methacrylic acid and ethylacrylate. In some embodiments, the polymer excipient is a copolymer of about 1.2:1 to about 1:1.2 methacrylic acid and ethylacrylate. In some embodiments, the polymer excipient is a copolymer of 1:1 methacrylic acid and ethylacrylate.

The polymer excipient may also act as a crystallization inhibitor, which helps slow or inhibit crystallization of TTI-101 when it is released from the formulation in the GI tract or mediates supersaturation stabilization (e.g., stabilization of a supersaturated solution).

In some embodiments, the solid amorphous dispersion comprises about 40% w/w to about 80% w/w of the compound of formula (I), or a pharmaceutically acceptable salt thereof. In some embodiments, the solid amorphous dispersion comprises about 40% w/w to about 70% w/w of the compound of formula (I), or a pharmaceutically acceptable salt thereof. In some embodiments, the solid amorphous dispersion comprises about 40% w/w to about 60% w/w of the compound of formula (I), or a pharmaceutically acceptable salt thereof. In some embodiments, the solid amorphous dispersion comprises about 45% w/w to about 55% w/w of the compound of formula (I), or a pharmaceutically acceptable salt thereof. In some embodiments, the solid amorphous dispersion comprises about 40% w/w of the compound of formula (I), or a pharmaceutically acceptable salt thereof. In some embodiments, the solid amorphous dispersion comprises about 45% w/w of the compound of formula (I), or a pharmaceutically acceptable salt thereof. In some embodiments, the solid amorphous dispersion comprises about 50% w/w of the compound of formula (I), or a pharmaceutically acceptable salt thereof. In some embodiments, the solid amorphous dispersion comprises about 55% w/w of the compound of formula (I), or a pharmaceutically acceptable salt thereof. In some embodiments, the solid amorphous dispersion comprises about 60% w/w of the compound of formula (I), or a pharmaceutically acceptable salt thereof. In some embodiments, the solid amorphous dispersion comprises about 65% w/w of the compound of formula (I), or a pharmaceutically acceptable salt thereof. In some embodiments, the solid amorphous dispersion comprises about 70% w/w of the compound of formula (I), or a pharmaceutically acceptable salt thereof. In some embodiments, the solid amorphous dispersion comprises about 75% w/w of the compound of formula (I), or a pharmaceutically acceptable salt thereof. In some embodiments, the solid amorphous dispersion comprises about 80% w/w of the compound of formula (I), or a pharmaceutically acceptable salt thereof.

In some embodiments, the solid amorphous dispersion comprises about 40% w/w to about 80% w/w of the polymer excipient. In some embodiments, the solid amorphous dispersion comprises In some embodiments, the solid amorphous dispersion comprises about 40% w/w to about 70% w/w of the polymer excipient. In some embodiments, the solid amorphous dispersion comprises about 40% w/w to about 60% w/w of the polymer excipient. In some embodiments, the solid amorphous dispersion comprises about 45% w/w to about 55% w/w of the polymer excipient. In some embodiments, the solid amorphous dispersion comprises about 40% w/w of the polymer excipient. In some embodiments, the solid amorphous dispersion comprises about 45% w/w of the polymer excipient. In some embodiments, the solid amorphous dispersion comprises about 50% w/w of the polymer excipient. In some embodiments, the solid amorphous dispersion comprises about 55% w/w of the polymer excipient. In some embodiments, the solid amorphous dispersion comprises about 60% w/w of the polymer excipient. In some embodiments, the solid amorphous dispersion comprises about 65% w/w of the polymer excipient. In some embodiments, the solid amorphous dispersion comprises about 70% w/w of the polymer excipient. In some embodiments, the solid amorphous dispersion comprises about 75% w/w of the polymer excipient. In some embodiments, the solid amorphous dispersion comprises about 80% w/w of the polymer excipient.

In some embodiments, provided herein are compositions comprising an effective amount of a solid amorphous dispersion described herein and at least one additional component. In some embodiments, the composition comprises about 5% w/w to about 50% w/w of the compound of formula (I), or a pharmaceutically acceptable salt thereof. In some embodiments, the composition comprises about 5% w/w to about 40% w/w of the compound of formula (I), or a pharmaceutically acceptable salt thereof. In some embodiments, the composition comprises about 10% w/w to about 40% w/w of the compound of formula (I), or a pharmaceutically acceptable salt thereof. In some embodiments, the composition comprises about 15% w/w to about 40% w/w of the compound of formula (I), or a pharmaceutically acceptable salt thereof. In some embodiments, the composition comprises about 20% w/w to about 40% w/w of the compound of formula (I), or a pharmaceutically acceptable salt thereof. In some embodiments, the composition comprises about 5% w/w to about 30% w/w of the compound of formula (I), or a pharmaceutically acceptable salt thereof. In some embodiments, the composition comprises about 10% w/w to about 30% w/w of the compound of formula (I), or a pharmaceutically acceptable salt thereof. In some embodiments, the composition comprises about 20% w/w to about 35% w/w of the compound of formula (I), or a pharmaceutically acceptable salt thereof. In some embodiments, the composition comprises about 25% w/w to about 35% w/w of the compound of formula (I), or a pharmaceutically acceptable salt thereof. In some embodiments, the composition comprises about 15% w/w to about 30% w/w of the compound of formula (I), or a pharmaceutically acceptable salt thereof. In some embodiments, the composition comprises about 15% w/w to about 25% w/w of the compound of formula (I), or a pharmaceutically acceptable salt thereof. In some embodiments, the composition comprises about 20% w/w to about 25% w/w of the compound of formula (I), or a pharmaceutically acceptable salt thereof. In some embodiments, the composition comprises about 25% w/w to about 30% w/w of the compound of formula (I), or a pharmaceutically acceptable salt thereof.

In some embodiments, the composition comprises about 5% w/w of the compound of formula (I), or a pharmaceutically acceptable salt thereof. In some embodiments, the composition comprises about 10% w/w of the compound of formula (I), or a pharmaceutically acceptable salt thereof. In some embodiments, the composition comprises about 15% w/w of the compound of formula (I), or a pharmaceutically acceptable salt thereof. In some embodiments, the composition comprises about 20% w/w of the compound of formula (I), or a pharmaceutically acceptable salt thereof. In some embodiments, the composition comprises about 25% w/w of the compound of formula (I), or a pharmaceutically acceptable salt thereof. In some embodiments, the composition comprises about 30% w/w of the compound of formula (I), or a pharmaceutically acceptable salt thereof. In some embodiments, the composition comprises about 35% w/w of the compound of formula (I), or a pharmaceutically acceptable salt thereof. In some embodiments, the composition comprises about 40% w/w of the compound of formula (I), or a pharmaceutically acceptable salt thereof.

In some embodiments, the composition comprises about 5% w/w to about 50% w/w of the polymer excipient. In some embodiments, the composition comprises about 5% w/w to about 40% w/w of the polymer excipient. In some embodiments, the composition comprises about 10% w/w to about 40% w/w of the polymer excipient. In some embodiments, the composition comprises about 15% w/w to about 40% w/w of the polymer excipient. In some embodiments, the composition comprises about 20% w/w to about 40% w/w of the polymer excipient. In some embodiments, the composition comprises about 5% w/w to about 30% w/w of the polymer excipient. In some embodiments, the composition comprises about 10% w/w to about 30% w/w of the polymer excipient. In some embodiments, the composition comprises about 20% w/w to about 35% w/w of the polymer excipient. In some embodiments, the composition comprises about 25% w/w to about 35% w/w of the polymer excipient. In some embodiments, the composition comprises about 15% w/w to about 30% w/w of the polymer excipient. In some embodiments, the composition comprises about 15% w/w to about 25% w/w of the polymer excipient. In some embodiments, the composition comprises about 20% w/w to about 25% w/w of the polymer excipient. In some embodiments, the composition comprises about 25% w/w to about 30% w/w of the polymer excipient.

In some embodiments, the composition comprises about 5% w/w of the polymer excipient. In some embodiments, the composition comprises about 10% w/w of the polymer excipient. In some embodiments, the composition comprises about 15% w/w of the polymer excipient. In some embodiments, the composition comprises about 20% w/w of the polymer excipient. In some embodiments, the composition comprises about 25% w/w of the polymer excipient. In some embodiments, the composition comprises about 30% w/w of the polymer excipient. In some embodiments, the composition comprises about 35% w/w of the polymer excipient. In some embodiments, the composition comprises about 40% w/w of the polymer excipient.

In some embodiments, the composition comprises 1:1 in w/w % of the compound of formula (I), or a pharmaceutically acceptable salt thereof to the polymer excipient. In some embodiments, the composition comprises 1:1.5 in w/w % of the compound of formula (I), or a pharmaceutically acceptable salt thereof to the polymer excipient. In some embodiments, the composition comprises 1.5:1 in w/w % of the compound of formula (I), or a pharmaceutically acceptable salt thereof to the polymer excipient. In some embodiments, the composition comprises 1:2 in w/w % of the compound of formula (I), or a pharmaceutically acceptable salt thereof to the polymer excipient. In some embodiments, the composition comprises 2:1 in w/w % of the compound of formula (I), or a pharmaceutically acceptable salt thereof to the polymer excipient.

In some embodiments, the composition comprises a crystallization inhibiting polymer as the additional component. In general, the crystallization inhibiting polymer helps slow or inhibit crystallization of the TTI-101 when it is released from the formulation in the GI tract, mediates supersaturation stabilization (e.g., stabilization of a supersaturated solution). Examples of crystallization inhibiting polymers include, but are not limited to, celluloses and derivatives thereof (e.g., microcrystalline cellulose, carboxymethylcellulose, hydroxypropyl methylcellulose (HPMC) or hypromellose, hydroxypropyl methylcellulose acetate succinate (HPMCAS) or hypromellose acetate succinate (e.g., HPMCAS-LG, HPMCAS-MG, HPMCAS-HG, HPMCAS-LF, HPMCAS-LMP), HPMC phthalate (HPMCP) (e.g., HPMCP-HP55, HPMCP-HP55S), Methocel™ (e.g., Methocel™ E3 (e.g., Methocel™ E3LV)), hydroxypropyl cellulose, hydroxyethylcellulose, ethylcellulose, and cellulose acetate phthalate).

In some embodiments, the crystallization inhibiting polymer is a methyl cellulose or HPMC. In some embodiments, the crystallization inhibiting polymer is HPMC. In some embodiments, the crystallization inhibiting polymer is HPMCAS (e.g., HPMCAS-LF, HPMCAS-LMP).

In some embodiments, the composition comprises about 1% w/w to about 40% w/w of the crystallization inhibiting polymer. In some embodiments, the composition comprises about 5% w/w to about 40% w/w of the crystallization inhibiting polymer. In some embodiments, the composition comprises about 10% w/w to about 40% w/w of the crystallization inhibiting polymer. In some embodiments, the composition comprises about 5% w/w to about 30% w/w of the crystallization inhibiting polymer. In some embodiments, the composition comprises about 15% w/w to about 35% w/w of the crystallization inhibiting polymer. In some embodiments, the composition comprises about 10% w/w to about 30% w/w of the crystallization inhibiting polymer. In some embodiments, the composition comprises about 15% w/w to about 40% w/w of the crystallization inhibiting polymer. In some embodiments, the composition comprises about 20% w/w to about 40% w/w of the crystallization inhibiting polymer. In some embodiments, the composition comprises about 5% w/w to about 30% w/w of the crystallization inhibiting polymer. In some embodiments, the composition comprises about 10% w/w to about 30% w/w of the crystallization inhibiting polymer. In some embodiments, the composition comprises about 15% w/w to about 30% w/w of the crystallization inhibiting polymer. In some embodiments, the composition comprises about 15% w/w to about 25% w/w of the crystallization inhibiting polymer. In some embodiments, the composition comprises about 20% w/w to about 30% w/w of the crystallization inhibiting polymer.

In some embodiments, the composition comprises about 1% w/w of the crystallization inhibiting polymer. In some embodiments, the composition comprises about 2% w/w of the crystallization inhibiting polymer. In some embodiments, the composition comprises about 3% w/w of the crystallization inhibiting polymer. In some embodiments, the composition comprises about 4% w/w of the crystallization inhibiting polymer. In some embodiments, the composition comprises about 5% w/w of the crystallization inhibiting polymer. In some embodiments, the composition comprises about 10% w/w of the crystallization inhibiting polymer. In some embodiments, the composition comprises about 15% w/w of the crystallization inhibiting polymer. In some embodiments, the composition comprises about 20% w/w of the crystallization inhibiting polymer. In some embodiments, the composition comprises about 25% w/w of the crystallization inhibiting polymer. In some embodiments, the composition comprises about 30% w/w of the crystallization inhibiting polymer. In some embodiments, the composition comprises about 35% w/w of the crystallization inhibiting polymer. In some embodiments, the composition comprises about 40% w/w of the crystallization inhibiting polymer.

In some embodiments, when the selected crystallization inhibiting polymer for the composition is the same as the polymer excipient, the composition comprises about 20% to about 60% w/w of the crystallization inhibiting polymer/polymer excipient. In some embodiments, when the crystallization inhibiting polymer for the composition is the same as the polymer excipient, the composition comprises about 20% to about 50% w/w of the crystallization inhibiting polymer/polymer excipient. In some embodiments, when the crystallization inhibiting polymer for the composition is the same as the polymer excipient, the composition comprises about 30% to about 60% w/w of the crystallization inhibiting polymer/polymer excipient. In some embodiments, when the crystallization inhibiting polymer for the composition is the same as the polymer excipient, the composition comprises about 30% to about 50% w/w of the crystallization inhibiting polymer/polymer excipient. In some embodiments, when the crystallization inhibiting polymer for the composition is the same as the polymer excipient, the composition comprises about 40% to about 60% w/w of the crystallization inhibiting polymer/polymer excipient. In some embodiments, when the crystallization inhibiting polymer for the composition is the same as the polymer excipient, the composition comprises about 40% to about 50% w/w of the crystallization inhibiting polymer/polymer excipient.

In some embodiments, when the crystallization inhibiting polymer for the composition is the same as the polymer excipient, the composition comprises about 20% w/w of the crystallization inhibiting polymer/polymer excipient. In some embodiments, when the crystallization inhibiting polymer for the composition is the same as the polymer excipient, the composition comprises about 30% w/w of the crystallization inhibiting polymer/polymer excipient. In some embodiments, when the crystallization inhibiting polymer for the composition is the same as the polymer excipient, the composition comprises about 40% w/w of the crystallization inhibiting polymer/polymer excipient. In some embodiments, when the crystallization inhibiting polymer for the composition is the same as the polymer excipient, the composition comprises about 50% w/w of the crystallization inhibiting polymer/polymer excipient. In some embodiments, when the crystallization inhibiting polymer for the composition is the same as the polymer excipient, the composition comprises about 60% w/w of the crystallization inhibiting polymer/polymer excipient.

In some embodiments, the composition further comprises an antioxidant.

In some embodiments, the antioxidant is vitamin E. In some embodiments, the antioxidant is ascorbyl palmitate. In some embodiments, the antioxidant is butylated hydroxytoluene. In some embodiments, the antioxidant is triethyl citrate. In some embodiments, the antioxidant is citric acid. In some embodiments, the antioxidant is ascorbic acid.

In some embodiments, the antioxidant is present in the composition in a % w/w of about 0.05 to about 5% (e.g., about 0.1 to about 3%, about 0.2 to about 1%). In some embodiments, the antioxidant is present in the composition in a % w/w of about 0.05%. In some embodiments, the antioxidant is present in the composition in a % w/w of about 0.1%. In some embodiments, the antioxidant is present in the composition in a % w/w of about 0.2%. In some embodiments, the antioxidant is present in the composition in a % w/w of about 0.3%. In some embodiments, the antioxidant is present in the composition in a % w/w of about 0.4%. In some embodiments, the antioxidant is present in the composition in a % w/w of about 0.5%. In some embodiments, the antioxidant is present in the composition in a % w/w of about 0.6%. In some embodiments, the antioxidant is present in the composition in a % w/w of about 0.7%. In some embodiments, the antioxidant is present in the composition in a % w/w of about 0.8%. In some embodiments, the antioxidant is present in the composition in a % w/w of about 0.9%. In some embodiments, the antioxidant is present in the composition in a % w/w of about 1%. In some embodiments, the antioxidant is present in the composition in a % w/w of about 2%. In some embodiments, the antioxidant is present in the composition in a % w/w of about 3%. In some embodiments, the antioxidant is present in the composition in a % w/w of about 4%. In some embodiments, the antioxidant is present in the composition in a % w/w of about 5%.

In some embodiments, the composition further comprises a surfactant.

In some embodiments, the surfactant is a copolymer comprising polyoxypropylene and polyoxyethylene, polyoxyl 40 hydrogenated castor oil, or a water-soluble derivative of natural Vitamin E (e.g., D-α-tocopheryl polyethylene glycol succinate (vitamin E TPGS)). In some embodiments, the surfactant is poloxamer 188, poloxamer 407, Kolliphor® RH40, or vitamin E TPGS.

In some embodiments, the surfactant is present in the composition in a % w/w of about 0.01 to about 10%. In some embodiments, the surfactant is present in the composition in a % w/w of about 0.01 to about 5%. In some embodiments, the surfactant is present in the composition in a % w/w of about 0.1 to about 5%. In some embodiments, the surfactant is present in the composition in a % w/w of about 0.5 to about 5%. In some embodiments, the surfactant is present in the composition in a % w/w of about 1 to about 5%. In some embodiments, the surfactant is present in the composition in a % w/w of about 1%. In some embodiments, the surfactant is present in the composition in a % w/w of about 2%. In some embodiments, the surfactant is present in the composition in a % w/w of about 3%. In some embodiments, the surfactant is present in the composition in a % w/w of about 4%. In some embodiments, the surfactant is present in the composition in a % w/w of about 5%.

In some embodiments, the composition comprises 150 mg of the compound of formula (I) or a pharmaceutically acceptable salt thereof. In some embodiments, the composition comprises at least 160 mg of the compound of formula (I) or a pharmaceutically acceptable salt thereof. In some embodiments, the composition comprises at least 170 mg of the compound of formula (I) or a pharmaceutically acceptable salt thereof. In some embodiments, the composition comprises at least 180 mg of the compound of formula (I) or a pharmaceutically acceptable salt thereof. In some embodiments, the composition comprises at least 190 mg of the compound of formula (I) or a pharmaceutically acceptable salt thereof. In some embodiments, the composition comprises at least 200 mg of the compound of formula (I) or a pharmaceutically acceptable salt thereof. In some embodiments, the composition comprises at least 220 mg of the compound of formula (I) or a pharmaceutically acceptable salt thereof. In some embodiments, the composition comprises at least 240 mg of the compound of formula (I) or a pharmaceutically acceptable salt thereof. In some embodiments, the composition comprises at least 260 mg of the compound of formula (I) or a pharmaceutically acceptable salt thereof. In some embodiments, the composition comprises at least 280 mg of the compound of formula (I) or a pharmaceutically acceptable salt thereof. In some embodiments, the composition comprises at least 300 mg of the compound of formula (I) or a pharmaceutically acceptable salt thereof.

In some embodiments, provided herein are solid oral dosage forms comprising a compound of formula (I) or a pharmaceutically acceptable salt thereof.

In some embodiments, provided herein are solid oral dosage forms comprising a solid amorphous dispersion or a composition described herein. In some embodiments, the oral dosage form is a tablet. In some embodiments, the oral dosage form comprises at least 100 mg of a compound of formula (I), or a pharmaceutically acceptable salt thereof. In some embodiments, the oral dosage form comprises at least 110 mg of a compound of formula (I), or a pharmaceutically acceptable salt thereof. In some embodiments, the oral dosage form comprises at least 120 mg of a compound of formula (I), or a pharmaceutically acceptable salt thereof. In some embodiments, the oral dosage form comprises at least 130 mg of a compound of formula (I), or a pharmaceutically acceptable salt thereof. In some embodiments, the oral dosage form comprises at least 140 mg of a compound of formula (I), or a pharmaceutically acceptable salt thereof. In some embodiments, the oral dosage form comprises at least 150 mg of a compound of formula (I), or a pharmaceutically acceptable salt thereof. In some embodiments, the oral dosage form comprises at least 160 mg of a compound of formula (I), or a pharmaceutically acceptable salt thereof. In some embodiments, the oral dosage form comprises at least 170 mg of a compound of formula (I), or a pharmaceutically acceptable salt thereof. In some embodiments, the oral dosage form comprises at least 180 mg of a compound of formula (I), or a pharmaceutically acceptable salt thereof. In some embodiments, the oral dosage form comprises at least 190 mg of a compound of formula (I), or a pharmaceutically acceptable salt thereof. In some embodiments, the oral dosage form comprises at least 200 mg of a compound of formula (I), or a pharmaceutically acceptable salt thereof. In some embodiments, the oral dosage form comprises at least 300 mg of a compound of formula (I), or a pharmaceutically acceptable salt thereof. In some embodiments, the oral dosage form comprises at least 400 mg of a compound of formula (I), or a pharmaceutically acceptable salt thereof. In some embodiments, the oral dosage form comprises at least 500 mg of a compound of formula (I), or a pharmaceutically acceptable salt thereof.

In some embodiments, the oral dosage form comprises about 200 mg of a compound of formula (I), or a pharmaceutically acceptable salt thereof. In some embodiments, the oral dosage form comprises about 250 mg of a compound of formula (I), or a pharmaceutically acceptable salt thereof. In some embodiments, the oral dosage form comprises about 300 mg of a compound of formula (I), or a pharmaceutically acceptable salt thereof. In some embodiments, the oral dosage form comprises about 350 mg of a compound of formula (I), or a pharmaceutically acceptable salt thereof. In some embodiments, the oral dosage form comprises about 400 mg of a compound of formula (I), or a pharmaceutically acceptable salt thereof. In some embodiments, the oral dosage form comprises about 450 mg of a compound of formula (I), or a pharmaceutically acceptable salt thereof. In some embodiments, the oral dosage form comprises about 500 mg of a compound of formula (I), or a pharmaceutically acceptable salt thereof.

In some embodiments, the composition further comprises at least one additional component. Said component can be intra-granular or extra-granular components that improve processability of the tablet manufacture (e.g., processability of roller compaction, tablet compression and film coating). The additional components can also impart good powder flow and compression characteristics to the material being compressed. Desirable characteristics of the additional components can include high-compressibility so as to allow for strong tablets to be made at low compression forces; good powder flow properties that can improve the powder flow of other excipients in the composition; and cohesiveness, for example to prevent a tablet from crumbling during processing, shipping, and handling. Other components which give physical characteristics to a finished tablet are coloring and flavoring agents (e.g., in the case of chewable tablets). Examples of additional components are described, for example, in the Handbook of Pharmaceutical Excipients (5^{th} edition), Edited by Raymond C Rowe, Paul J. Sheskey, and Sian C. Owen; Publisher: Pharmaceutical Press.

In some embodiments, the additional components include, but are not limited to, diluents, binders, fillers, disintegrants, surfactants, lubricants, flavoring agents, and colors. The additional component can serve multiple functions. For example, a diluent may also serve as a filler. As another example, a surfactant may also serve as a lubricant.

In some embodiments, diluents or fillers are added, for example, to increase the bulk weight of the blend resulting in a practical size for compression. Diluents or fillers that may be used include one or more of calcium salts such as calcium phosphate dibasic and sugars such as lactose, sucrose, dextrose, microcrystalline cellulose, mannitol, and maltodextrin. Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. In some embodiments, the diluent or filler is microcrystalline cellulose, which can be manufactured by the controlled hydrolysis of alpha-cellulose. In some embodiments, suitable microcrystalline cellulose has an average particle size of from about 20 nm to about 200 nm. Suitable microcrystalline cellulose includes Avicel PH 101, Avicel PH 102, Avicel PH 103, Avicel PH 105 and Avicel PH 200. In some embodiments, the diluent or filler is silicified microcrystalline cellulose, such as ProSolv® SMCC 50 or ProSolv® SMCC HD 90. In some embodiments, the diluent or filler is lactose. In some embodiments, the diluent or filler is a mixture of two or more diluents or fillers. In some embodiments, the diluent or filler is microcrystalline cellulose, silicified microcrystalline cellulose, or powdered cellulose, or a mixture thereof.

In some embodiments, the amount of diluent or filler employed in the composition is from about 5% to about 50% w/w. In some embodiments, the amount of diluent or filler employed in the composition is from about 5% to about 40% w/w. In some embodiments, the amount of diluent or filler employed in the composition is from about 5% to about 30% w/w. In some embodiments, the amount of diluent or filler employed in the composition is from about 5% to about 25% w/w. In some embodiments, the amount of diluent or filler employed in the composition is from about 5% to about 20% w/w. In some embodiments, the amount of diluent or filler employed in the composition is from about 5% to about 15% w/w. In some embodiments, the amount of diluent or filler employed in the composition is from about 5% to about 10% w/w. In some embodiments, the amount of diluent or filler employed in the composition is from about 10% to about 50% w/w. In some embodiments, the amount of diluent or filler employed in the composition is from about 10% to about 40% w/w. In some embodiments, the amount of diluent or filler employed in the composition is from about 10% to about 30% w/w. In some embodiments, the amount of diluent or filler employed in the composition is from about 10% to about 20% w/w. In some embodiments, the amount of diluent or filler employed in the composition is from about 15% to about 40% w/w. In some embodiments, the amount of diluent or filler employed in the composition is from about 15% to about 35% w/w. In some embodiments, the amount of diluent or filler employed in the composition is from about 15% to about 30% w/w. In some embodiments, the amount of diluent or filler employed in the composition is from about 15% to about 20% w/w. In some embodiments, the amount of diluent or filler employed in the composition is from about 20% to about 50% w/w. In some embodiments, the amount of diluent or filler employed in the composition is from about 20% to about 40% w/w. In some embodiments, the amount of diluent or filler employed in the composition is from about 20% to about 35% w/w. In some embodiments, the amount of diluent or filler employed in the composition is from about 5% to about 20% w/w. In some embodiments, the amount of diluent or filler employed in the composition is from about 20% to about 30% w/w. In some embodiments, the amount of diluent or filler employed in the composition is about 5% w/w. In some embodiments, the amount of diluent or filler employed in the composition is about 10% w/w. In some embodiments, the amount of diluent or filler employed in the composition is about 15% w/w. In some embodiments, the amount of diluent or filler employed in the composition is about 20% w/w. In some embodiments, the amount of diluent or filler employed in the composition is about 25% w/w. In some embodiments, the amount of diluent or filler employed in the composition is about 30% w/w. In some embodiments, the amount of diluent or filler employed in the composition is about 35% w/w. In some embodiments, the amount of diluent or filler employed in the composition is about 40% w/w. In some embodiments, the amount of diluent or filler employed in the composition is about 45% w/w. In some embodiments, the amount of diluent or filler employed in the composition is about 50% w/w.

In some embodiments, the composition further includes a lubricant. Lubricants are typically added to prevent the tableting materials from clumping together and from stick to the tablet punches, minimize friction during tablet compression, and to allow for removal of the compressed tablet from the die. Such lubricants are included in the final tablet mix in amounts usually less than 5 by weight per weight of a composition. Examples of lubricants include, but are not limited to, colloidal silica, magnesium trisilicate, talc, magnesium carbonate, stearic acid, magnesium oxide, glycerylbehaptate, polyethylene glycol, ethylene oxide polymers, sodium lauryl sulphate, magnesium stearate, aluminum stearate, calcium stearate, sodium stearyl fumarate, stearic acid, magnesium lauryl stearate, and mixtures of magnesium stearate with sodium lauryl sulphate. In some embodiments, the lubricant is sodium lauryl sulfate or sodium stearyl fumarate, or a mixture thereof. In some embodiments, the lubricant is a mixture of two or more lubricants.

In some embodiments, the amount of lubricant employed in the composition is from about 0.01 to about 5.0% w/w. In some embodiments, the amount of lubricant employed in the composition is from about 0.01 to about 4.0% w/w. In some embodiments, the amount of lubricant employed in the composition is from about 0.01 to about 3.0% w/w. In some embodiments, the amount of lubricant employed in the composition is from about 0.1 to about 3.0% w/w. In some embodiments, the amount of lubricant employed in the composition is from about 0.01 to about 2.0% w/w. In some embodiments, the amount of lubricant employed in the composition is from about 0.05 to about 2.0% w/w. In some embodiments, the amount of lubricant employed in the composition is from about 0.1 to about 2.0% w/w. In some embodiments, the amount of lubricant employed in the composition is from about 0.01 to about 1.5% w/w. In some embodiments, the amount of lubricant employed in the composition is from about 0.05 to about 1.5% w/w. In some embodiments, the amount of lubricant employed in the composition is from about 0.1 to about 1.5% w/w. In some embodiments, the amount of lubricant employed in the composition is from about 0.5 to about 4.0% w/w. In some embodiments, the amount of lubricant employed in the composition is from about 0.5 to about 3.5% w/w. In some embodiments, the amount of lubricant employed in the composition is from about 0.5 to about 3.0 w/w. In some embodiments, the amount of lubricant employed in the composition is from about 0.5 to about 2.5% w/w. In some embodiments, the amount of lubricant employed in the composition is from about 0.5 to about 2.0% w/w. In some embodiments, the amount of lubricant employed in the composition is from about 0.5 to 1.5% w/w. In some embodiments, the amount of lubricant employed in the composition is from about 1.0 to about 3.0% w/w. In some embodiments, the amount of lubricant employed in the composition is from about 1.0 to about 2.5% w/w. In some embodiments, the amount of lubricant employed in the composition is from about 1.0 to about 2.0% w/w. In some embodiments, the amount of lubricant employed in the composition is from about 1.5 to about 2.0% w/w. In some embodiments, the amount of lubricant employed in the composition is from about 0.5 to 1.0% w/w. In some embodiments, the amount of lubricant employed in the composition is about 0.1% w/w. In some embodiments, the amount of lubricant employed in the composition is about 0.2% w/w. In some embodiments, the amount of lubricant employed in the composition is about 0.3% w/w. In some embodiments, the amount of lubricant employed in the composition is about 0.4% w/w. In some embodiments, the amount of lubricant employed in the composition is about 0.5% w/w. In some embodiments, the amount of lubricant employed in the composition is about 0.6% w/w. In some embodiments, the amount of lubricant employed in the composition is about 0.7% w/w. In some embodiments, the amount of lubricant employed in the composition is about 0.8% w/w. In some embodiments, the amount of lubricant employed in the composition is about 0.9% w/w. In some embodiments, the amount of lubricant employed in the composition is about 1.0% w/w. In some embodiments, the amount of lubricant employed in the composition is about 1.5% w/w. In some embodiments, the amount of lubricant employed in the composition is about 1.8% w/w. In some embodiments, the amount of lubricant employed in the composition is about 2.0% w/w. In some embodiments, the amount of lubricant employed in the composition is about 2.5% w/w.

In some embodiments, glidants are added, for example, to improve tablet powder's flowability by reducing interparticle friction and cohesion. Examples of glidants include magnesium stearate, magnesium carbonate, silica (e.g., colloidal silicon dioxide (such as the grades sold as Aerosil)), starch and talc. Glidants may be present in the composition at an amount of from 0.01 to about 5% w/w. In some embodiments, the amount of glidant employed in the composition is from about 0.01 to about 4.0% w/w. In some embodiments, the amount of glidant employed in the composition is from about 0.01 to about 3.0% w/w. In some embodiments, the amount of glidant employed in the composition is from about 0.01 to about 2.0% w/w. In some embodiments, the amount of glidant employed in the composition is from about 0.05 to about 2.0% w/w. In some embodiments, the amount of glidant employed in the composition is from about 0.1 to about 2.0% w/w. In some embodiments, the amount of glidant employed in the composition is from about 0.01 to about 1.5% w/w. In some embodiments, the amount of glidant employed in the composition is from about 0.05 to about 1.5% w/w. In some embodiments, the amount of glidant employed in the composition is from about 0.1 to about 1.5% w/w. In some embodiments, the amount of glidant employed in the composition is from about 0.5 to 1.5% w/w. In some embodiments, the amount of glidant employed in the composition is from about 0.5 to 1.0% w/w. In some embodiments, the amount of glidant employed in the composition is about 0.1% w/w. In some embodiments, the amount of glidant employed in the composition is about 0.2% w/w. In some embodiments, the amount of glidant employed in the composition is about 0.3% w/w. In some embodiments, the amount of glidant employed in the composition is about 0.4% w/w. In some embodiments, the amount of glidant employed in the composition is about 0.5% w/w. In some embodiments, the amount of glidant employed in the composition is about 0.6% w/w. In some embodiments, the amount of glidant employed in the composition is about 0.7% w/w. In some embodiments, the amount of glidant employed in the composition is about 0.8% w/w. In some embodiments, the amount of glidant employed in the composition is about 0.9% w/w. In some embodiments, the amount of glidant employed in the composition is about 1.0% w/w. In some embodiments, the amount of glidant employed in the composition is about 2.0% w/w.

In some embodiments, a tablet disintegrant is present in the composition at an amount, for example, to expedite dissolution (e.g., increase the rate of tablet disintegration). Disintegrants are excipients which can oppose the physical forces of particle bonding in a tablet when the dosage form is placed in an aqueous environment. Disintegrants include starch derivatives and salts of carboxymethylcellulose. Examples of pharmaceutically acceptable disintegrants include, but are not limited to, starches, e.g., sodium starch glycolate, pregelatinized starch; clays; celluloses; alginates; gums; cross-linked polymers, e.g., cross-linked polyvinyl pyrrolidone (e.g., polyvinyl polypyrrolidone, PVPP, crospovidone, crospolividone), cross-linked calcium carboxymethylcellulose and cross-linked sodium carboxymethylcellulose (sodium croscarmellose); and polysaccharides. In some embodiments, the disintegrant is a mixture of two or more disintegrants.

Generally the amount of disintegrant can be from 0.1 to about 25% w/w of the composition. In some embodiments, the amount of disintegrant is from about 1% to about 15% w/w of the composition. In some embodiments, the amount of disintegrant is from about 1% to about 10% w/w of the composition. In some embodiments, the amount of disintegrant is from about 1% to about 8% w/w of the composition. In some embodiments, the amount of disintegrant is from about 5% to about 10% w/w of the composition. In some embodiments, the amount of disintegrant is from about 3% to about 10% w/w of the composition. In some embodiments, the amount of disintegrant is from about 3% to about 8% w/w of the composition. In some embodiments, the amount of disintegrant is from about 5% to about 10% w/w of the composition. In some embodiments, the amount of disintegrant is from about 5% to about 15% w/w of the composition. In some embodiments, the amount of disintegrant is from about 1% to about 5% w/w of the composition. In some embodiments, the amount of disintegrant is about 1% w/w of the composition. In some embodiments, the amount of disintegrant is about 2% w/w of the composition. In some embodiments, the amount of disintegrant is about 3% w/w of the composition. In some embodiments, the amount of disintegrant is about 4% w/w of the composition. In some embodiments, the amount of disintegrant is about 5% w/w. In some embodiments, the amount of disintegrant is about 6% w/w of the composition. In some embodiments, the amount of disintegrant is about 7% w/w of the composition. In some embodiments, the amount of disintegrant is about 8% w/w of the composition. In some embodiments, the amount of disintegrant is about 9% w/w of the composition. In some embodiments, the amount of disintegrant is about 10% w/w of the composition. In some embodiments, the amount of disintegrant is about 15% w/w of the composition.

In some embodiments, a formulation is set forth as in Table 2 or Table 3 (e.g., wherein the total wt. % in the composition does not exceed 100%).

TABLE 2
Exemplary Formulations

| | | | |
|---|---|---|---|
| | Formulation 6 | Formulation 7 | Formulation 8 |
| Component | (w/w) | (w/w) | (w/w) |

Spray-Dried Dispersion - Solid Amorphous Dispersion

| | | | |
|---|---|---|---|
| TTI-101 | 10-30% | 15-25% | 22% |
| Polymer excipient | 10-30% | 15-25% | 22% |

Intra-granular Blend

| | | | |
|---|---|---|---|
| Diluent/Filler | 5-20% | 10-15% | 12% |
| Disintegrant | 0.5-5% | 1-5% | 3.2% |
| Surfactant/ | 0.1-2% | 0.5-1.0% | 0.8% |
| Lubricant | | | |
| CIP | 5-25% | 10-20% | 15% |
| CIP | 1-10% | 5-10% | 6% |

Extra-granular Blend

| | | | |
|---|---|---|---|
| Diluent/Filler | 5-25% | 10-20% | 15% |
| Disintegrant | 0.5-10% | 1-5% | 3% |
| Lubricant | 0.1-2% | 0.5-1.5% | 1% |

TABLE 3
Exemplary Formulations

| | Formulation | Formulation | Formulation |
|---|---|---|---|
| Component | 9 (w/w) | 10 (w/w) | 11 (w/w) |
| TTI-101 | 10-30% | 15-25% | 22% |
| Polymer excipient | 10-30% | 15-25% | 22% |
| Diluent/Filler | 15-40% | 20-35% | 27% |
| Disintegrant | 1-10% | 3-8% | 6% |
| Surfactant/Lubricant | 0.1-2% | 0.5-1.5% | 1% |
| CIP | 10-30% | 15-25% | 21% |
| Lubricant | 0.1-2% | 0.5-1.5% | 1% |

Tablets may be plain, film, sugar coated, bisected, embossed, layered, and/or sustained-release. They can be made in a variety of sizes, shapes, and colors. Tablets may be swallowed, chewed, or dissolved in the buccal cavity or beneath the tongue.

The tablets of the present invention may be coated or otherwise compounded to provide a dosage form affording the advantage of prolonged action, protect tablet ingredients from deterioration, make large or unpleasant-tasking tablets easier to swallow, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

In certain embodiments, the tablet is polymer-coated. In certain embodiments, the tablet is coated with Opadry® EZ white.

In certain embodiments, the compositions described herein is administered as a suspension in a pharmaceutically acceptable carrier (e.g., after, optionally, crushing the compositions or solid oral dosage forms into fine powder for the suspension). In certain embodiments, the suspension is administered orally. In certain embodiments, the suspension is administered via a feeding tube (e.g., via percutaneous endoscopic gastronomy (PEG) tube or G tube). PEG allows for a feeding tube to be place through the abdominal wall and into the stomach, allowing the composition to be put directly into the stomach, bypassing the mouth and esophagus.

### Methods of Preparing Solid Amorphous Dispersions

Disclosed herein, in certain embodiments, are methods of preparing a solid amorphous dispersion of a compound of Formula I, comprising:
i) dissolving the compound of formula (I),

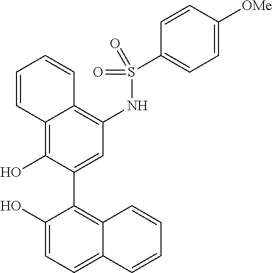 [SMILES: COc1ccc(S(=O)(=O)Nc2cc(-c3c(O)ccc4ccccc34)c(O)c3ccccc23)cc1]

or a pharmaceutically acceptable salt thereof, and a polymer excipient in a solvent to form a solution; and
ii) atomizing the solution of step (i) into a drying chamber in a spray dried dispersion apparatus to prepare the solid amorphous dispersion.

In some embodiments, the method further comprises dissolving a crystallization inhibiting polymer in step (i).

### Methods of Use

Also provided herein, in certain embodiments, are methods of using a composition comprising a solid amorphous dispersion comprising a compound of Formula (I) and a polymer excipient, such as in therapeutic or other methods described herein. In some embodiments, the methods involve the use of (e.g., comprise the administration of) a compound of Formula (I), wherein the compound of Formula (I) is formulated in a manner described herein (e.g., is present in a composition as described herein). In some embodiments, a compound of Formula (I) (e.g., as formulated herein e.g., as an oral dosage form) is utilized in a method for treating a disease or disorder mediated by STAT3, or a disease or disorder that is otherwise treatable with a STAT3 inhibitor. In specific embodiments, provided herein are methods of treating cancer. In other specific embodiments, provided herein are methods of treating fibrosis. In still other specific embodiments, provided herein are methods of treating an inflammatory disease/disorder.

Provided in certain embodiments herein are methods of treating, preventing, or reducing the risk or severity of a cancer in an individual in need thereof, the method comprising administering to the individual any composition or oral dosage described herein. In some embodiments, the cancer treated according to a method provided herein is a liver cancer, lung cancer, head and neck cancer, breast cancer, skin cancer, kidney cancer, testicular cancer, colon cancer, rectal cancer, gastric cancer, skin cancer, metastatic melanoma, prostate cancer, ovarian cancer, cervical cancer, bone cancer, spleen cancer, gall bladder cancer, brain cancer, pancreatic cancer, stomach cancer, anal cancer, prostate cancer, multiple myeloma, post-transplant lymphoproliferative disease, restenosis, myelodysplastic syndrome, leukemia, lymphoma, or acute myelogenous leukemia. In some embodiments, a cancer treated according to a method provided herein is a liver cancer, lung cancer, liver carcinoma, hepatocellular carcinoma, head and neck squamous cell carcinoma, non-small cell lung cancer, or estrogen receptor-positive breast cancer. In some embodiments, a cancer treated according to a method provided herein is head and neck cancer, lung cancer, liver cancer, breast cancer, ovarian cancer, colon cancer, multiple myeloma, leukemia, or pancreatic cancer. In some embodiments, the leukemia is acute myelogenous leukemia. In some embodiments, the gastric cancer is gastric adenocarcinoma.

Provided in certain embodiments herein are methods of treating, preventing, or reducing the risk or severity of an inflammatory disease/disorder in an individual in need thereof, the method comprising administering to the individual any composition or oral dosage described herein. In some embodiments, the inflammatory disease/disorder treated herein is inflammatory bowel disease (IBD), ulcerative colitis, Crohn's disease, asthma, anaphylaxis, cancer cachexia, chronic kidney disease cachexia, nonalcoholic steatohepatitis (NASH), psoriasis, uveitis, scleritis, multiple sclerosis, or pancreatitis. In some embodiments, inflammation treated herein is inflammatory bowel disease (IBD), ulcerative colitis, Crohn's disease, asthma, anaphylaxis, cancer cachexia, chronic kidney disease cachexia, or nonalcoholic steatohepatitis (NASH). In some embodiments, the anaphylaxis comprises anaphylactic shock.

Provided in certain embodiments herein are methods of treating, preventing, or reducing the risk or severity of a fibrotic disease/disorder in an individual in need thereof, the method comprising administering to the individual any composition or oral dosage described herein. In certain embodiments, the fibrotic disease/disorder is skin fibrosis (or dermal fibrosis), cardiac fibrosis, cirrhosis, pulmonary fibrosis, bone marrow fibrosis, intestine fibrosis, pancreatic fibrosis, joint fibrosis, liver fibrosis, retroperitoneum, renal fibrosis, myelofibrosis, non-alcoholic fatty liver disease, steatohepatitis, systemic sclerosis (including diffuse systemic sclerosis or limited systemic sclerosis), endomyocardial fibrosis, myocardial infarction, atrial fibrosis, mediastinal fibrosis, progressive massive fibrosis, nephrogenic systemic fibrosis, Keloid, arthrofibrosis, adhesive capsulitis, or cystic fibrosis. In certain embodiments, the fibrosis is skin fibrosis (scleroderma), cardiac fibrosis, cirrhosis, pulmonary fibrosis, bone marrow fibrosis, intestine fibrosis, pancreatic fibrosis, joint fibrosis, liver fibrosis, retroperitoneum, myelofibrosis, non-alcoholic fatty liver disease, steatohepatitis, or systemic sclerosis. In certain embodiments, the fibrosis is skin fibrosis (scleroderma), cardiac fibrosis, cirrhosis, or pulmonary fibrosis.

In certain embodiments, the fibrotic disease/disorder is fibrosis following exposure to certain drugs such as chemotherapy, fibrosis following exposure to environmental or other toxins or allergens, fibrosis occurring after an ischemia/reperfusion injury such as myocardial infarction or hypotension, fibrosis occurring after radiation, fibrosis following hepatitis induced by alcohol, toxins, drugs or infections, primary biliary cirrhosis, fibrosis following viral infections involving the heart, liver, or lung, and/or idiopathic retroperitoneal fibrosis.

Provided in certain embodiments herein are methods of treating, preventing, or reducing the risk or severity of a muscle wasting disease/disorder, muscle weakness disease/disorder, or cachexia in an individual in need thereof, the method comprising administering to the individual any composition or oral dosage described herein. The muscle weakness and/or muscle wasting and/or cachexia may have an unknown cause or it may be associated with an underlying condition. The underlying condition may be a catabolic condition. In some embodiments, the underlying medical condition associated with cachexia is least renal disease or failure, cancer, AIDS, HIV infection, chronic obstructive lung disease (including emphysema), multiple sclerosis, congestive heart failure, tuberculosis, familial amyloid polyneuropathy, acrodynia, hormonal deficiency, metabolic acidosis, infectious disease, chronic pancreatitis, autoimmune disorder, celiac disease, Crohn's disease, electrolyte imbalance, Addison's disease, sepsis, burns, trauma, fever, long bone fracture, hyperthyroidism, prolonged steroid therapy, surgery, bone marrow transplant, atypical pneumonia, brucellosis, endocarditis, Hepatitis B, lung abscess, mastocytosis, paraneoplastic syndrome, polyarteritis nodosa, sarcoidosis, systemic lupus erythematosus, myositis, polymyositis, dematomyosytis, rheumatological diseases, autoimmune disease, collagen-vascular disease, visceral leishmaniasis, prolonged bed rest, and/or addiction to drugs, such as amphetamine, opiates, or barbitutates.

Provided in certain embodiments herein are methods of treating, preventing, or reducing the risk or severity of an allergic reaction in an individual in need thereof, the method comprising administering to the individual any composition, or oral dosage described herein. In some embodiments, the allergic reaction is induced following an exposure to an allergen. In some embodiments, the allergen is a food allergen (such as milk, legumes, shellfish, tree nuts, eggs, fish, soy, and wheat), an environmental allergen or seasonal allergen (such as pollen or mold), a venom allergen (such as from wasp, bee, ant, hornet, yellow jacket, or asp), a medication allergen (such as anesthetics, β-lactam antibiotics, aspirin, non-steroidal anti-inflammatory drug, chemotherapy, vaccine, protamine, or herbal preparations), or latex. In some embodiments, the allergic reaction is anaphylaxis, anaphylactic shock, allergic rhinitis, urticaria, food allergy, drug allergy, hymenoptera allerga, bronchial constriction, asthma, or eczema.

STAT3 also plays an important role in viral infection and pathogenesis (Chang Z, et al. STAT3 roles in viral infection: antiviral or proviral?. Future Virol. 2018; 13 (8): 557-574). Provided in certain embodiments herein are methods of treating, preventing, or reducing the risk or severity of a viral infection in an individual in need thereof, the method comprising administering to the individual any composition or oral dosage described herein. In some embodiments, the viral infection is a chronic viral infection. In some embodiments, the chronic viral infection is AIDS, HIV infection, Hepatitis B infection, Hepatitis C virus infection, or Epstein-Barr virus infection.

Provided in certain embodiments herein are methods of treating, preventing, or reducing the risk or severity of graft-versus-host diseases, pulmonary lymphangioleiomyomatosis, chagasic cardiomyopathy, age-related macular degeneration, amyloidosis, astrogliosis in Alzheimer's or other neurodegenerative diseases, or familial amyloid polyneuropathy.

Provided in certain embodiments herein are methods of treating, preventing, or reducing the risk or severity of a neurodegenerative disease. In some embodiments, the neurodegenerative disease is chemotherapy-induced peripheral neuropathy, diabetic neuropathy, or chemobrain. Provided in certain embodiments herein are methods of treating, preventing, or reducing the risk or severity of pain in an individual in need thereof, the method comprising administering to the individual any composition or oral dosage described herein. In some embodiments, pain is neuropathic pain.

Provided in certain embodiments herein are methods of treating, preventing, or reducing the risk or severity of insulin resistance in an individual in need thereof, the method comprising administering to the individual any composition or oral dosage described herein. In some embodiments, the insulin resistance is a result of an underlying condition. In some embodiments, the insulin resistance is associated with muscle of the individual being treated. In some embodiments, the insulin resistance is caused by any reason for the individual, such as elevated free fatty acids in the blood, obesity, being overweight, having visceral fat, having a high fructose intake, having inflammation, being inactive, dysbiosis of the gut microbiota, and/or being genetically predisposed. In certain embodiments, any method provided herein is a method of treating or reducing the risk or severity of medical conditions associated with insulin resistance or that are complications of insulin resistance at least in part, such as severe high blood sugar; severe low blood sugar; heart attack; stroke; kidney disease (including chronic, for example, chronic kidney disease (CKD)); eye problems; cancer; non-alcoholic fatty liver disease (NAFLD); polycystic ovarian syndrome (PCOS); metabolic syndrome; diabetes; or Alzheimer's disease, for example. In certain embodiments, the insulin resistance is a hallmark of metabolic syndrome and type 2 diabetes. Metabolic syndrome is a group of risk factors associated with type 2 diabetes and heart disease. Its symptoms include high blood triglycerides, blood pressure, belly fat, and blood sugar, as well as low HDL (good) cholesterol levels.

In some embodiments, the pill burden discussed herein is associated with any suitable therapeutic (e.g., daily) dose of compound of Formula (I) and/or loading of compound of Formula (I) in the oral dosage form(s), such as any dose or amount described herein.

In certain embodiments, the method comprises administering at least 1 mg/kg/day of the compound of formula (I) to the individual. In certain embodiments, the method comprises administering at least 10 mg/kg/day of the compound of formula (I) to the individual. In certain embodiments, the method comprises administering at least 20 mg/kg/day of the compound of formula (I) to the individual. In certain embodiments, the method comprises administering at least 25 mg/kg/day of the compound of formula (I) to the individual.

In some embodiments, a number of adverse events is reduced with SDD formulations as compared to SEDD formulations. In some embodiments, the severity of adverse events is reduced with the SDD formulations as compared to the SEDD formulations. In some embodiments, a number of individuals experiencing an adverse event is reduced with the SDD formulations as compared to the SEDD formulations. In some embodiments, the adverse event is diarrhea.

### EXAMPLES

### Example 1. Polymer and Surfactant Screening for Solid Amorphous Dispersions of TTI-101

Liquid-liquid phase separation (LLPS) was used to identify polymers that would prevent the crystallization and precipitation of supersaturated solutions of TTI-101 solutions in fasted-state simulated intestinal fluid (FaSSIF). Polymers that were screened in this study include: HPMC AS-MPP, HPMC AS-HG, HPMC AS-LG, HPMC-3, PVPVA64, and Eudragit L100. HPMC-based polymers showed good performance, sustaining the super-saturated concentration of TTI-101 throughout the experiment (240 minutes). Eudragit L100 showed a similar performance for the initial 50 minutes.

In addition, mixtures of HPMC AS-LG/HPMC-E3+/−5% w/w surfactants and HPMAC AS-LG+/−5% w/w surfactants were prepared and evaluated with LLPS. Surfactants used in this study include: Poloxamer 188, Poloxamer 407, Kolliphor RH40, and vitamin E TPGS. Addition of the poloxamer 407, Kolliphor RH40 and vitamin E TPGS provided marginal improvement in LLPS performance for the mixture of HPMC AS-LG/HPMC-E3.

### Example 2. TTI-101 Spray Dried Dispersions (SDDs)

Particle size reduction for TTI-101 by micronization and nano-milling provided poor bioavailability (e.g., 10-20 times lower bioavailability than a solution of TTI-101). Hot melt extrusion (HME) was used and the resulting dispersions were evaluated using DSC miscibility, DSC extrudability and rheology studies. Polymer systems tried include: Soluplus; Soluplus+Kolliphor RH40; Soluplus+Vitamin E TPGS; and PVPVA64+Kolliphor RH40. Dispersions of TTI-101 made using HME showed high TTI-101 degradation and performance in biorelevant in-vitro super-saturated kinetic dissolution.

To prevent TTI-101 degradation, SDD technique was used to produce compositions comprising TTI-101. Exemplary SDD compositions were prepared with the polymer system and concentration of TTI-101 as shown in Table 4.

TABLE 4
Exemplary SDD Compositions

| | % w/w TTI-101 | | |
|---|---|---|---|
| | in polymer | | |
| Composition | system | Polymer system | SDD solvent |
| 1 | 50 | HPMC-E3 | DCM:MeOH |
| | | | (80:20) |
| 2 | 50 | HPMC AS-LG | DCM:MeOH |
| | | | (80:20) |
| 3 | 50 | HPMC AS-LG + 5% | DCM:MeOH |
| | | Poloxomer 407 | (80:20) |
| 4 | 50 | HPMC AS-GD + 5% | DCM:MeOH |
| | | vitamin E TPGS | (80:20) |
| 5 | 67 | HPMC AS-LG | DCM:MeOH |
| | | | (80:20) |
| 6 | 20 | Eudragit EPO | Acetone or |
| | | | Acetone:Water |
| | | | (90:10) |
| 7 | 50 | Eudragit L100-55 | Acetone or |
| | | | Acetone:Water |
| | | | (90:10) |
| 8 | 50 | Plasdone S630 | Acetone or |
| | | Ultra | Acetone:Water |
| | | | (90:10) |
| 9 | 50 | HPMC AS-LG | Acetone or |
| | | | Acetone:Water |
| | | | (90:10) |
| 10 | 50 | HPMC AS-LG + 5% | Acetone or |
| | | vitamin E TPGS | Acetone:Water |
| | | | (90:10) |
| 11 | 60 | Eudragit L100-55 | Acetone or |
| | | | Acetone:Water |
| | | | (90:10) |
| 12 | 70 | Eudragit L100-55 | Acetone or |
| | | | Acetone:Water |
| | | | (90:10) |
| 13 | 80 | Eudragit L100-55 | Acetone or |
| | | | Acetone:Water |
| | | | (90:10) |
| 14 | 60 | HPMC AS-LG | Acetone or |
| | | | Acetone:Water |
| | | | (90:10) |
| 15 | 70 | HPMC AS-LG | Acetone or |
| | | | Acetone:Water |
| | | | (90:10) |
| 16 | 80 | HPMC AS-LG | Acetone or |
| | | | Acetone:Water |
| | | | (90:10) |

Compositions 1-5 exhibited marginal performance in super-saturated kinetic dissolution (SSKD). Characterization of compositions 1-5 showed that they were not fully amorphous. On the other hand, compositions 6-10 were fully amorphous. Addition of a crystallization inhibiting polymer (CIP) such as HPMC AS-LG to the compositions improved peak concentration and supersaturation of TTI-101. Compositions 11 and 12 exhibited good stability while composition 13 exhibited two glass transition temperatures (Tg's) indicating phase separation in the SDD and also showed TTI-101 crystallization during the stability assessment. Composition 14 showed changes in Tg during stability assessment and compositions 15 and 16 exhibited two Tg's indicating a phase separation in the SDD.

Other CIPs that were used include: MC-A4M, HPMC-E4M, and HPMC-K4M. Compositions with MC-A4M performed similarly to the compositions with HPMC AS-LG as the CIP.

### Example 3. Antioxidant Screening for TTI-101 SDD-Based Compositions

Antioxidants such as citric acid, ascorbic acid, or ascorbyl palmitate (each 1% w/w) were added to the SDD compositions of TTI-101 with HPMC AS-LG or Eudragit L100-55. The resulting compositions exhibited changes in Tg during stability assessment and provided marginal improvements in chemical stability of the SDD-based compositions.

### Example 4. CIP Screening for TTI-101 SDD-Based Compositions

CIPs were added to the SDD compositions of TTI-101 with HPMC AS-LG or Eudragit L100-55. CIPs that were screened include: HPMC AS-LG, MC-A4M, HPMC-E4M, and HPMC-K4M. Compositions with MC-A4M performed similarly to the compositions with HPMC AS-LG as the CIP. HPMC AS-LG concentrations were: 10%, 15% and 25% w/w (ratio of HPMC AS-LG (CIP) to SDD. The composition with 25% w/w HPMC AS-LG to SDD showed highest TTI-101 concentration and a steady rise in TTI-101 with no drop in the concentration throughout the run (6 hours) (see FIGURE).

### Example 5. Tablet Formulations

An exemplary tablet formulation was prepared with a SDD of TTI-101 and methacrylic acid and ethyl acrylate copolymer as the polymer excipient (Table 5). The formulation was mixed, roller compacted (RC), milled, blended, compressed and coated with a film to form an exemplary tablet. Intra and extra-granular components were added to improve processability of roller compaction, tablet compression, and film coating.

TABLE 5
Example 200 mg TTI-101 Tablet Formulation

| | | | |
|---|---|---|---|
| | | | Quantity |
| Component | Function | % w/w | per Unit |

Spray-Dried Dispersion

| | | | | |
|---|---|---|---|---|
| TTI-101 | Active ingredient | 22.28% | 200 | mg |
| Methacrylic Acid and | Polymer | 22.28% | 200 | mg |
| Ethyl Acrylate | | | | |

| | |
|---|---|
| Copolymer | Excipient |

Intra-granular Blend

| Silicified Microcrystalline | Diluent/Filler | 12.15% | 109.1 | mg |
|---|---|---|---|---|
| Cellulose | | | | |
| Croscarmellose Sodium | Disintegrant | 3.24% | 29.1 | mg |
| Sodium Lauryl Sulfate | Surfactant/ | 0.81% | 7.3 | mg |
| | Lubricant | | | |
| Hypromellose acetate | Crystallization | 14.58% | 130.9 | mg |
| succinate LMP | inhibitor | | | |
| Hypromellose acetate | Crystallization | 5.67% | 50.9 | mg |

| | |
|---|---|
| succinate LF | inhibitor |

| | | | |
|---|---|---|---|
| Total: | 81.00% | 727.3 | mg |

Extra-granular Blend

| | | | | |
|---|---|---|---|---|
| Silicified Microcrystalline | Diluent/Filler | 15.00% | 134.7 | mg |
| Cellulose | | | | |
| Croscarmellose Sodium | Disintegrant | 3.00% | 26.9 | mg |
| Sodium Stearyl Fumarate | Lubricant | 1.00% | 9.0 | mg |

| | | | |
|---|---|---|---|
| Total: | 100.00% | 897.9 | mg |

Coating

| | | | | |
|---|---|---|---|---|
| Opadry EZ White | Coating film | 3.0% of | 26.9 | mg |

core

### Example 6. Pharmacokinetics and Bioavailability Study of TTI-101 SDD-Based Formulations

In this study, the pharmacokinetics and oral bioavailability of the SDD-based formulations were evaluated in rats. Formulations A-D were prepared according to Tables 6-7. Formulation E was prepared according to the methods described in WO2021150912.

TABLE 6
Exemplary Formulations A-B

| | Amount | Weight | | Amount | Weight |
|---|---|---|---|---|---|
| Component | (% w/w) | (mg) | Component | (% w/w) | (mg) |
| Formulation A - | | | Formulation B - | | |
| intragranular blend/RC blend | | | intragranular blend/RC blend | | |
| TTI-101:Eudragit | 55.00 | 825 | TTI-101:Eudragit | 65.00 | 975 |
| L100-55: (50:50) | | | L100-55: (50:50) | | |
| HPMC AS LF | 25.00 | 375 | HPMC AS LF | 15.00 | 225 |
| Prosolve | 15.00 | 225 | Prosolve | 15.00 | 225 |
| HD 90 | | | HD 90 | | |
| Croscarmellose | 4.00 | 60 | Croscarmellose | 4.00 | 60 |
| Sodium | | | Sodium (Ac-Di-Sol) | | |
| SLS (sodium | 1.00 | 15 | SLS | 1.00 | 15 |
| lauryl sulfate) | | | | | |
| Total | 100.00 | 1500 | Total | 100.00 | 1500 |

TABLE 7
Exemplary Formulations C-D

| | Amount | Weight | | Amount | Weight |
|---|---|---|---|---|---|
| Component | (% w/w) | (mg) | Component | (% w/w) | (mg) |
| Formulation C | | | Formulation D | | |
| Intragranular (IG) blend | | | Intragranular (IG) blend | | |
| TTI-101:Eudragit | 55.00 | 825 | TTI-101:Eudragit | 55.00 | 825 |
| L100-55: (50:50) | | | L100-55: (50:50) | | |
| HPMC AS LF | 25.00 | 375 | HPMC AS LG | 25.00 | 375 |
| Prosolve HD 90 | 15.00 | 225 | Prosolve HD 90 | 15.00 | 225 |
| Croscarmellose | 4.00 | 60 | Croscarmellose | 4.00 | 60 |
| Sodium | | | Sodium (Ac-Di-Sol) | | |
| SLS | 1.00 | 15 | SLS | 1.00 | 15 |
| Total | 100.00 | 1500 | Total | 100.00 | 1500 |
| Extragranular blend | | | Extragranular blend | | |
| IG granules | 81.00 | 1215 | IG granules | 81.00 | 1215 |
| Prosolve HD 90 | 15.00 | 225 | Prosolve HD 90 | 15.00 | 225 |
| Croscarmellose | 3.00 | 45 | Croscarmellose | 3.00 | 45 |
| Sodium | | | Sodium (Ac-Di-Sol) | | |
| SSF (Sodium | 1.00 | 15 | SSF (Sodium | 1.00 | 15 |
| stearyl fumarte) | | | stearyl fumarte) | | |
| Total | 100.00 | 1500.00 | Total | 100.00 | 1500.00 |

Table 8 shows exemplary dosages of TTI-101 administered to rats and % TTI-101 in each formulation.

TABLE 8
Administration of TTI-101 Formulations in Rats

| Formulation/route of | | % TTI-101 in |
|---|---|---|
| administration | Dosage (mg/kg) | formulation (w/w) |
| A (PO) | 25 | 27.5 |
| B (PO) | 25 | 32.5 |
| C (PO) | 25 | 22.3 |
| D (PO) | 25 | 22.3 |
| E (PO) | 25 | 7.2 |
| F (IV) | 25 | N/A |

Formulation F (IV) was prepared in DMSO/EtOH/PEG400/Saline at May 2, 1943/50% v/v (TTI-101 5 mg/mL). The absolute oral bioavailability compared to Formulation F for the oral formulations were 0.183, 0.156, 0.229, 0.139, and 0.173, respectively for Formulations A-E. The Cmax was: Formulation F (77000 ng/ml), Formulation E (10300 ng/ml), Formulation C (5500 ng/mL), Formulation D (4490 ng/ml), Formulation A (4060 ng/mL), and Formulation B (3500 ng/mL). The AUClast was Formulation F (188000 h*ng/mL), Formulation C (41800 h*ng/mL), Formulation E (31400 h*ng/mL), Formulation A (30700 h*ng/ml), Formulation B (28600 h*ng/ml), and Formulation D (25800 h*ng/ml).

The above results show that a formulation comprising composition 7 of Example 2 with a CIP such as HPMC AS LF showed higher bioavailability to a capsule formulation (Formulation E) of TTI-101 and a formulation comprising composition 7 of Example 2 with a CIP such as HPMC AS LG showed similar bioavailability to Formulation E of TTI-101 (KolliphorRH40:PEG600:Polysorbate 20:Labrasol:Citric Acid:TTI-101 about 27.1:38.8:10.8:13.5:0.5:9.2). Formulation E is a capsule formulation comprising 80 mg of TTI-101, whereas exemplary tablet formulations described herein comprises 200 mg TTI-101.

### Example 7. Oral STAT3 Inhibitor TTI-101 in Patients with Advanced Cancers and in Healthy Volunteers

In one Phase I trial, TTI-101 was assessed in patients with advanced cancers. In another trial, TTI-101 was assessed in healthy volunteers.

Briefly, Formulations 1-3 were prepared as in the previous examples. Two trials were performed. The first trial was performed similar to the trial outlined below in Table 9 (for patients with advanced cancers) and was made up of a first cohort of subjects receiving Formulation 1, a second cohort of subjects receiving Formulation 2, and a third cohort of subjects receiving Formulation 3. The second trial was similar to the trial outlined below in Table 10 (for healthy volunteers) and was made up of a cohort of subjects receiving Formulation 3.

TABLE 9
Trial Information for TTI-101 in Patients with Advanced Cancers
Study Information

| | |
|---|---|
| Study Type | Interventional |
| Study Phase | Phase 1 |
| Study Design | Allocation: Non-Randomized |
| | Intervention Model: Single Group Assignment |
| | Masking: None (Open Label) |
| | Primary Purpose: Treatment |
| Condition | Breast Cancer |
| | Head and Neck Squamous Cell Carcinoma |
| | Non Small Cell Lung Cancer |
| | Hepatocellular Cancer |
| | Colorectal Cancer |
| | Gastric Adenocarcinoma |
| | Melanoma |
| | Advanced Cancer |
| Intervention | Drug: TTI-101 Oral capsule |
| | Drug: TTI-101 Oral tablet |
| Study Arms | Experimental: Dose escalation study |
| | Participants will receive up to 4 dose levels of TTI-101 to |
| | determine RP2D |
| | Intervention: Drug: TTI-101 |
| | Experimental: Dose expansion study |
| | Enrollment in the dose expansion may commence with approval |
| | from the safety review committee. Participants will be enrolled |
| | and treated at the RP2D of TTI-101 |
| | Intervention: Drug: TTI-101 |
| | Experimental: Food effect study |
| | Participants will be treated with TTI-101 at the RP2D under fed |
| | and fasted conditions to assess the bioavailability of TTI-101 and |
| | to determine the best conditions for taking the study drug |
| | Intervention: Drug: TTI-101 |
| | Experimental: Dose expansion, cross-over study |
| | Participants will be administered different formulations of TTI- |
| | 101 to compare bioavailability. |
| | Interventions: |
| | Drug: TTI-101 - Formulation 1 |
| | Drug: TTI-101 - Formulation 2 |
| | Drug: TTI-101 - Formulation 3 |

Outcomes

| | |
|---|---|
| Primary Outcome | Maximum Tolerated Dose of TTI-101 |
| Measures | To determine the maximum tolerated dose (MTD), dose-limiting |
| | toxicities, and tolerability of TTI-101 administered orally to |
| | patients with advanced breast cancer and other solid tumors. |
| | Dose-limiting toxicity is defined as a Grade 3 or above adverse |
| | event (using CTCAE v5.0) within the first treatment cycle (28- |
| | days). |
| | Pharmacokinetics - Cmax |
| | Pharmacokinetics - Tmax |
| | Pharmacokinetics - AUC(0-t) |
| Secondary Outcome | Pharmacodynamics of TTI-101 in patients |
| Measures | Levels of pY-STAT3 measured before and before and after |
| | receiving TTI-101 will be measured. |
| | Complete Response (CR) - Target Lesions |
| | Complete Response (CR): Disappearance of all target lesions. |
| | Partial Response (PR) - Target Lesions |
| | Partial Response (PR): At least a 30% decrease in the sum of |
| | diameters of target lesions, taking as reference the baseline sum |
| | diameters. |
| | Progressive Disease (PD) - Target Lesions |
| | Progressive Disease (PD): At least a 20% increase in the sum of |
| | diameters of target lesions. |
| | Stable Disease (SD) - Target Lesions |
| | Stable Disease (SD): Neither sufficient shrinkage to qualify for PR |
| | nor sufficient increase to qualify for PD, taking as reference the |
| | smallest sum diameters while on study. |
| | Complete Response (CR) - Non-target Lesions |
| | Complete Response (CR): Disappearance of all non-target lesions |
| | and normalization of tumor marker level. |
| | Non-CR/Non-PD - Non-target Lesions |
| | Non-CR/Non-PD: Persistence of one or more non-target lesion(s) |
| | and/or maintenance of tumor marker level above the normal |
| | limits. |
| | Progressive Disease (PD) - Non-target Lesions |
| | Progressive Disease (PD): Unequivocal progression of existing |
| | non-target lesions. |
| | Best Overall Response |
| Other Pre-specified | Explore association between biomarkers and antitumor efficacy |
| Outcome Measures | and survival outcome based on RECIST 1.1 for uHCC patients. |
| | Assess the association between STAT3 inhibition, fibrosis (if |
| | applicable), antitumor activity and survival outcomes after |
| | receiving TTI-101. Tissue and blood immune monitoring will be |
| | based on 2 biopsies. Association between biomarkers including |
| | pY-STAT3, PD1, and PD-L1 proteins expression by IHC, gene |
| | expression profiling, and antitumor efficacy and survival outcome |
| | of TTI-101 based on RECIST 1.1. |
| | Assess the effect of food on bioavailability |
| | Assess the effect of food on bioavailability of TTI-101 in the dose |
| | expansion phase |
| | Assess the bioavailability between different formulations of TTI- |
| | 101 |
| | Assess the bioavailability between different formulations of TTI- |
| | 101 in the dose expansion phase |

TABLE 10
Trial Information for TTI-101 in Healthy Volunteers
Study Information

| | |
|---|---|
| Study Type | Interventional |
| Study Phase | Phase 1 |
| Study Design | This is a Phase 1, single-center, single-sequence, drug-drug interaction |
| | study to evaluate the safety, tolerability, and pharmacokinetics of TTI- |
| | 101 in steady state with single oral doses of nintedanib (Part 1) and |
| | pirfenidone (Part 2) in healthy participants. |
| Condition | Healthy Volunteers |
| Intervention | Drug: TTI-101 (Formulation 3) |
| | Drug: Nintedanib |
| | Drug: Pirfenidone |
| Study Design | Part 1: Participants will receive a single oral dose of nintedanib on |
| | Day 1, repeated oral dosing of TTI-101 (200 mg tablets) on Day 3 |
| | to Day 18, and single oral doses of nintedanib on Day 5 and Day 17. |
| | Part 2: Participants will receive a single oral dose of pirfenidone |
| | on Day 1, repeated oral dosing of TTI-101 (200 mg tablets) on |
| | Day 3 to Day 18, and single oral doses of pirfenidone on Day 5 |
| | and Day 17. |
| | Oral dosing of TTI-101, nintedanib, and/or pirfenidone will be |
| | done in the fed state, after completion of a low-fat meal. |

Outcomes

| | |
|---|---|
| Primary | To assess the safety and tolerability of repeated oral doses of TTI-101 when |
| Objective | dosed in combination with a single dose of nintedanib (Part 1) or |
| | pirfenidone (Part 2) in healthy participants. |
| Secondary | To evaluate the pharmacokinetics of single doses of nintedanib (Part 1) or |
| Objective | pirfenidone (Part 2) when dosed alone, or when dosed in combination with |
| | TTI-101 in steady state in healthy participants. |
| Outcome | Safety and tolerability, including 12-lead electrocardiograms (ECGs). |
| Measures | Systemic pharmacokinetics (parent; metabolites): |
| | Day 1 to Day 3: nintedanib (Part 1) or pirfenidone (Part 2) area under |
| | the plasma concentration-time curve (AUC) up to time t, where t is |
| | the last point with concentrations above the lower limit of |
| | quantification (AUC₀₋ₜ), AUC from time 0 to infinity (AUC_{0-inf}), |
| | maximum observed plasma concentration (Cₘₐₓ), time to attain |
| | maximum observed plasma concentration (tₘₐₓ), terminal elimination |
| | rate constant (kₑₗ), terminal elimination half-life (t_{1/2}), apparent oral |
| | clearance (CL/F), and apparent volume of distribution (V/F) |
| | Day 3: TTI-101 AUC from time 0 to 12 hours (AUC_{0-12 h}), Cₘₐₓ, tₘₐₓ; |
| | kₑₗ, t_{1/2}, CL/F, and V/F, if possible |
| | Day 5 and Day 17: TTI-101 AUC_{0-12 h}; Cₘₐₓ, tₘₐₓ; CL/F, V/F, kₑₗ, and |
| | t_{1/2}, if possible |
| | Days 5 to 7, and Days 17 to 19: nintedanib (Part 1) or pirfenidone |
| | (Part 2) AUC₀₋ₜ, AUC_{0-inf}, Cₘₐₓ, tₘₐₓ, kₑₗ, t_{1/2}, CL/F, and V/F |
| | Day 12: TTI-101 predose (trough) level |

The most common treatment-related adverse event observed with the SEDD formulation (Formulation 2) was diarrhea, which was reported among 44.7% of the 47 patients treated with TTI-101 SEDD (Table 11). A total of 48 patients across both studies were treated with the SDD formulation (Formulation 3) of TTI-101, and as shown in the table below, fewer patients reported diarrhea (6.3%) as compared to the SEDD formulation, with no Grade 2 or Grade 3 diarrhea events. Patients treated with Formulation 3 also had lower diarrhea events than those dosed with the Labrasol/PEG formulation (Formulation 1).

TABLE 11
Treatment-related adverse events observed with Formulations 1-3

| Preferred | Formulation 1 | Formulation 2 | Formulation 3 |
|---|---|---|---|
| Term/Grade | N = 15 | N = 47 | N = 48 |
| Diarrhea | 6 (40.0%) | 21 (44.7%) | 3 (6.3%) |
| Grade 1: Mild | 1 (6.7%) | 13 (27.7) | 3 (6.3%) |
| Grade 2: Moderate | 2 (13.3%) | 3 (6.4%) | 0 (0.0%) |
| Grade 3: Severe | 3 (20.0%) | 5 (10.6%) | 0 (0.0%) |

In addition, the pharmacokinetic data from the healthy volunteers' study (dosed with Formulation 3), in comparison to the Phase 1 study (dosed with Formulation 2) demonstrates the mean exposures are equivalent. Further, patient exposures at every time point in the curve exceeded the IC90 for STAT3 dependent growth (data not shown).

In sum, Formulation 3 (prepared by the SDD technique) lowers the pill burden required to achieve a therapeutic dose of TTI-101. For example, Formulation 3 can deliver 200 mg of TTI-101 per unit versus 80 mg of TTI-101 per unit when Formulation 2 is used and 30 mg of TTI-101 per unit when Formulation 1 is used. Formulation 3 has reduced rate and severity of treatment emergent adverse events, and has similar exposures to Formulation 2.

1. A solid amorphous dispersion comprising:
a) a compound of formula (I): 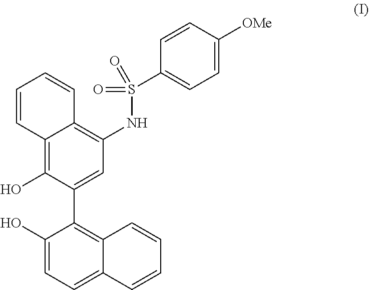 [SMILES: COc1ccc(S(=O)(=O)Nc2cc(-c3c(O)ccc4ccccc34)c(O)c3ccccc23)cc1]
or a pharmaceutically acceptable salt thereof; and
b) a polymer excipient.

2. The solid amorphous dispersion of claim 1, comprising about 40% w/w to about 80% w/w of the compound of formula (I), or a pharmaceutically acceptable salt thereof in the dispersion.

3. The solid amorphous dispersion of claim 1, comprising about 40% w/w to about 70% w/w of the compound of formula (I), or a pharmaceutically acceptable salt thereof in the dispersion.

4. The solid amorphous dispersion of claim 1, comprising about 40% w/w to about 60% w/w of the compound of formula (I), or a pharmaceutically acceptable salt thereof in the dispersion.

5. The solid amorphous dispersion of claim 1, comprising about 45% w/w to about 55% w/w of the compound of formula (I), or a pharmaceutically acceptable salt thereof in the dispersion.

6. The solid amorphous dispersion of claim 1, comprising about 50% w/w of the compound of formula (I), or a pharmaceutically acceptable salt thereof in the dispersion.

7. The solid amorphous dispersion of claim 1, comprising about 60% w/w of the compound of formula (I), or a pharmaceutically acceptable salt thereof in the dispersion.

8. The solid amorphous dispersion of claim 1, comprising about 70% w/w of the compound of formula (I), or a pharmaceutically acceptable salt thereof in the dispersion.

9. The solid amorphous dispersion of any one of claims 1-8, wherein the polymer excipient is hydroxypropyl methylcellulose (HPMC), a copolymer comprising methacrylic acid, a copolymer comprising vinyl acetate and N-vinyl-2-pyrrolidone.

10. The solid amorphous dispersion of any one of claims 1-9, wherein the polymer excipient is hydroxypropyl methylcellulose acetate succinate (HPMC AS).

11. The solid amorphous dispersion of claim 10, wherein HPMC AS is HPMC AS L grade (HPMC AS-L).

12. The solid amorphous dispersion of any one of claims 1-8, wherein the polymer excipient is a copolymer comprising methacrylic acid and ethylacrylate, a copolymer comprising methacrylic acid and methyl methacrylate, or a copolymer comprising N,N-dimethylaminoethyl methacrylate, methylmethacrylate, and butylmethacrylate.

13. The solid amorphous dispersion of claim 12, wherein the polymer excipient is a copolymer comprising methacrylic acid and ethylacrylate.

14. The solid amorphous dispersion of claim 13, wherein the polymer excipient is a copolymer of about 1.4:1 to about 1:1.4 methacrylic acid and ethylacrylate.

15. The solid amorphous dispersion of claim 13, wherein the polymer excipient is a copolymer of about 1.2:1 to about 1:1.2 methacrylic acid and ethylacrylate.

16. The solid amorphous dispersion of claim 13, wherein the polymer excipient is a copolymer of 1:1 methacrylic acid and ethylacrylate.

17. The solid amorphous dispersion of any one of claims 1-16, further comprising a crystallization inhibiting polymer.

18. The solid amorphous dispersion of claim 17, wherein the crystallization inhibiting polymer (CIP) is a methyl cellulose or HPMC.

19. The solid amorphous dispersion of claim 18, wherein the crystallization inhibiting polymer is HPMC.

20. The solid amorphous dispersion of claim 19, wherein the crystallization inhibiting polymer is HPMC AS.

21. The solid amorphous dispersion of claim 20, wherein HPMC AS is HPMC AS-LF or HPMC AS-LMP, or a mixture thereof.

22. The solid amorphous dispersion of any one of claims 17-21, comprising about 5% w/w to about 30% w/w of the crystallization inhibiting polymer in the dispersion.

23. The solid amorphous dispersion of any one of claims 17-22, comprising about 10% w/w to about 30% w/w of the crystallization inhibiting polymer in the dispersion.

24. The solid amorphous dispersion of any one of claims 17-22, comprising about 20% w/w to about 30% w/w of the crystallization inhibiting polymer in the dispersion.

25. The solid amorphous dispersion of any one of claims 17-22, comprising about 25% w/w of the crystallization inhibiting polymer in the dispersion.

26. A solid amorphous dispersion comprising:
a) a compound of formula (I): 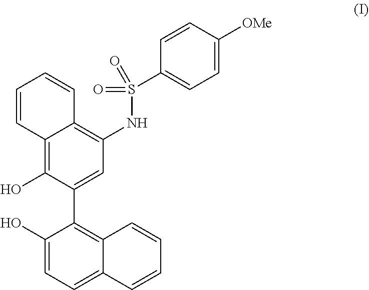 [SMILES: COc1ccc(S(=O)(=O)Nc2cc(-c3c(O)ccc4ccccc34)c(O)c3ccccc23)cc1]
or a pharmaceutically acceptable salt thereof, in an amount of about 40% w/w to about 60% w/w of the dispersion; and
b) a polymer excipient in an amount of about 40% to about 60% w/w of the dispersion.

27. The solid amorphous dispersion of claim 26, comprising about 45% w/w to about 55% w/w of the compound of formula (I), or a pharmaceutically acceptable salt thereof in the dispersion.

28. The solid amorphous dispersion of claim 26, comprising about 50% w/w of the compound of formula (I), or a pharmaceutically acceptable salt thereof in the dispersion.

29. The solid amorphous dispersion of any one of claims 26-28, wherein the polymer excipient is hydroxypropyl methylcellulose (HPMC), a copolymer comprising methacrylic acid, a copolymer comprising vinyl acetate and N-vinyl-2-pyrrolidone.

30. The solid amorphous dispersion of any one of claims 26-29, wherein the polymer excipient is hydroxypropyl methylcellulose acetate succinate (HPMC AS).

31. The solid amorphous dispersion of claim 30, wherein HPMC AS is HPMC AS L grade (HPMC AS-L).

32. The solid amorphous dispersion of any one of claims 26-29, wherein the polymer excipient is a copolymer comprising methacrylic acid and ethylacrylate, a copolymer comprising methacrylic acid and methyl methacrylate, or a copolymer comprising N,N-dimethylaminoethyl methacrylate, methylmethacrylate, and butylmethacrylate.

33. The solid amorphous dispersion of claim 32, wherein the polymer excipient is a copolymer comprising methacrylic acid and ethylacrylate.

34. The solid amorphous dispersion of claim 33, wherein the polymer excipient is a copolymer of about 1.4:1 to about 1:1.4 methacrylic acid and ethylacrylate.

35. The solid amorphous dispersion of claim 33, wherein the polymer excipient is a copolymer of about 1.2:1 to about 1:1.2 methacrylic acid and ethylacrylate.

36. The solid amorphous dispersion of claim 33, wherein the polymer excipient is a copolymer of 1:1 methacrylic acid and ethylacrylate.

37. The solid amorphous dispersion of any one of claims 26-36, comprising about 50% w/w of the polymer excipient in the dispersion.

38. The solid amorphous dispersion of any one of claims 26-37, further comprising a crystallization inhibiting polymer.

39. The solid amorphous dispersion of claim 38, wherein the crystallization inhibiting polymer is HPMC.

40. The solid amorphous dispersion of claim 39, wherein HPMC is HPMC AS.

41. The solid amorphous dispersion of any one of claims 37-40, wherein the HPMC AS is HPMC AS-LF or HPMC AS-LMP, or a mixture thereof.

42. The solid amorphous dispersion of any one of claims 1-41, further comprising an antioxidant.

43. A composition comprising a solid amorphous dispersion comprising:
a) at least 150 mg of a compound of formula (I): 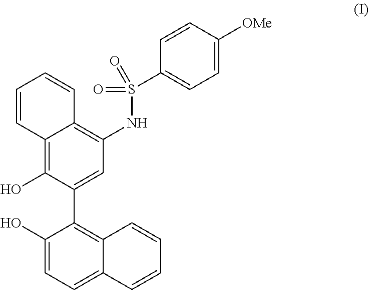 [SMILES: COc1ccc(S(=O)(=O)Nc2cc(-c3c(O)ccc4ccccc34)c(O)c3ccccc23)cc1]
or a pharmaceutically acceptable salt thereof; and
b) a polymer excipient.

44. The composition of claim 43, further comprising a diluent or filler.

45. The composition of claim 44, wherein the diluent or filler is a mixture of two or more diluents or fillers.

46. The composition of claim 45, wherein the diluent or filler is in an amount of about 20-35% w/w of the composition.

47. The composition of claim 45 or 46, wherein the diluent or filler is in an amount of about 15-40% w/w of the composition.

48. The composition of any one of claims 44-47, wherein the diluent or filler is microcrystalline cellulose, silicified microcrystalline cellulose, or powdered cellulose.

49. The composition of any one of claims 43-48, further comprising a disintegrant.

50. The composition of claim 49, wherein the disintegrant is a mixture of two or more diluents.

51. The composition of claim 50, wherein the disintegrant is in an amount of about 3-8% w/w of the composition.

52. The composition of claim 50, wherein the disintegrant is in an amount of about 1-10% w/w of the composition.

53. The composition of any one of claims 49-52, wherein the disintegrant is croscarmellose sodium.

54. The composition of any one of claims 43-53, further comprising a lubricant.

55. The composition of claim 54, wherein the lubricant is a mixture of two or more lubricants.

56. The composition of claim 55, wherein the lubricant is an amount of about 0.5-4% w/w of the composition.

57. The composition of claim 55, wherein the lubricant is an amount of about 1-3% w/w of the composition.

58. The composition of any one of claims 54-57, wherein the lubricant is sodium lauryl sulphate, magnesium stearate, or sodium stearyl fumarate.

59. The composition of any one of claims 43-58, further comprising a crystallization inhibiting polymer.

60. The composition of claim 59, wherein the crystallization inhibiting polymer is in an amount of about 10-30% w/w of the composition.

61. The composition of claim 59, wherein the crystallization inhibiting polymer is in an amount of about 15-25% w/w of the composition.

62. A composition comprising:
a) a solid amorphous dispersion comprising:
i) at least 150 mg of a compound of formula (I): 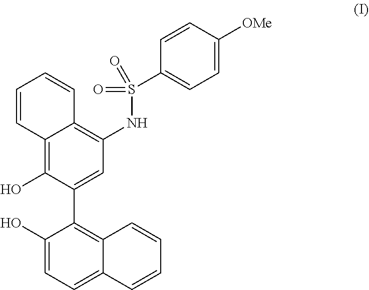 [SMILES: COc1ccc(S(=O)(=O)Nc2cc(-c3c(O)ccc4ccccc34)c(O)c3ccccc23)cc1]
or a pharmaceutically acceptable salt thereof; and
ii) a polymer excipient in an amount of about 20-35% w/w of the composition;
b) a crystallization inhibiting polymer in an amount of about 15-35% w/w of the composition;
c) a diluent or filler in an amount of about 5-25% of the composition;
d) a disintegrant in an amount of about 1-8% w/w of the composition; and
e) a lubricant in an amount of about 0.1-3% w/w of the composition.

63. A composition comprising:
a) a solid amorphous dispersion comprising
i) at least 150 mg of a compound of formula (I): 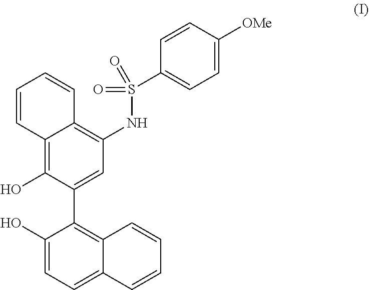 [SMILES: COc1ccc(S(=O)(=O)Nc2cc(-c3c(O)ccc4ccccc34)c(O)c3ccccc23)cc1]
or a pharmaceutically acceptable salt thereof; and
ii) a polymer excipient in an amount of about 15-25% w/w of the composition;
b) a crystallization inhibiting polymer in an amount of about 15-25% w/w of the composition;
c) a diluent or filler in an amount of about 20-35% of the composition;
d) a disintegrant in an amount of about 3-8% w/w of the composition; and
e) a lubricant in an amount of about 1-3% w/w of the composition.

64. A solid oral dosage form comprising a therapeutically effective amount of a compound of formula (I) or a pharmaceutically acceptable salt thereof.

65. A solid oral dosage form comprising a solid amorphous dispersion of any one of claims 1-43 or a composition of any one of claims 44-63.

66. The oral dosage form of claim 64 or 65, wherein the oral dosage form is a tablet.

67. The oral dosage form of claim 66, further comprising a coating film.

68. A method of treating cancer in an individual in need thereof, the method comprising administering to the individual the oral dosage form of any one of claims 64-67.

69. The method of claim 68, wherein the cancer is head and neck cancer, lung cancer, liver cancer, breast cancer, skin cancer, kidney cancer, testicular cancer, colon cancer, rectal cancer, gastric cancer, skin cancer, metastatic melanoma, prostate cancer, ovarian cancer, cervical cancer, bone cancer, spleen cancer, gall bladder cancer, brain cancer, pancreatic cancer, stomach cancer, anal cancer, prostate cancer, multiple myeloma, post-transplant lymphoproliferative disease, restenosis, myelodysplastic syndrome, leukemia, or lymphoma.

70. The method of claim 68, wherein the cancer is head and neck cancer, lung cancer, liver cancer, breast cancer, ovarian cancer, colon cancer, multiple myeloma, prostate cancer, cervical cancer, brain cancer, pancreatic cancer, myelodysplastic syndrome, leukemia, lymphoma, neuroblastoma, kidney cancer, skin cancer, or metastatic melanoma.

71. The method of claim 68, wherein the cancer is head and neck cancer, lung cancer, liver cancer, breast cancer, ovarian cancer, colon cancer, multiple myeloma, leukemia or pancreatic cancer.

72. A method of treating fibrosis in an individual in need thereof, the method comprising administering to the individual the oral dosage form of any one of claims 64-67.

73. The method of claim 72, wherein the fibrosis is pulmonary fibrosis, bone marrow fibrosis, intestine fibrosis, pancreatic fibrosis, joint fibrosis, liver fibrosis, retroperitoneum, kidney fibrosis, myelofibrosis, dermal fibrosis, non-alcoholic fatty liver disease, steatohepatitis, or systemic sclerosis.

74. A method of treating an inflammatory disease or disorder in an individual in need thereof, the method comprising administering to the individual the oral dosage form of any one of claims 64-67.

75. The method of claim 74, wherein the inflammatory disease or disorder is inflammatory bowel disease, ulcerative colitis, psoriasis, uveitis, scleritis, multiple sclerosis, pancreatitis, or asthma.

76. The method of claim 74, wherein the inflammatory disease or disorder is inflammatory bowel disease, ulcerative colitis, asthma, or psoriasis.

77. A method of treating chemotherapy-induced peripheral neuropathy, diabetic neuropathy, or familial amyloid polyneuropathy in an individual in need thereof, the method comprising administering to the individual the oral dosage form of any one of claims 64-67.

78. A method of treating cachexia in an individual in need thereof, the method comprising administering to the individual the oral dosage form of any one of claims 64-67.

79. A method of treating anaphylaxis in an individual in need thereof, the method comprising administering to the individual the oral dosage form of any one of claims 64-67.

80. The method of any one of claims 68-79, comprising administering at least 1 mg/kg/day of the compound of formula (I) to the individual.

81. The method of any one of claims 68-79, comprising administering at least 10 mg/kg/day of the compound of formula (I) to the individual.

82. The method of any one of claims 68-79, comprising administering at least 20 mg/kg/day of the compound of formula (I) to the individual.

83. The method of any one of claims 68-79, comprising administering at least 25 mg/kg/day of the compound of formula (I) to the individual.

84. A method of preparing a solid amorphous dispersion comprising
a) a compound of formula (I): 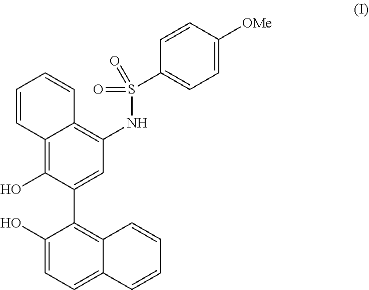 [SMILES: COc1ccc(S(=O)(=O)Nc2cc(-c3c(O)ccc4ccccc34)c(O)c3ccccc23)cc1]
or a pharmaceutically acceptable salt thereof, and
b) a polymer excipient, the method comprising:
i) dissolving the compound of formula (I), or a pharmaceutically acceptable salt thereof, and the polymer excipient in a solvent to form a solution; and
ii) atomizing the solution of step (i) into a drying chamber in a spray dried dispersion apparatus to prepare the solid amorphous dispersion.

85. The method of claim 84, further comprising dissolving a crystallization inhibiting polymer in step (i).

86. A composition prepared according to the method of claim 84 or 85.

87. An oral dosage form comprising an effective amount of a composition of claim 86.
